(19) United States
(12) Patent Application Publication
(10) Pub. No.: US 2019/0060361 A1
(43) Pub. Date: Feb. 28, 2019

(54) METHODS AND COMPOSITIONS RELATING TO REGULATORY T CELLS

(71) Applicant: THE BRIGHAM AND WOMEN'S HOSPITAL, INC., Boston, MA (US)

(72) Inventor: Guo-Ping SHI, Newton, MA (US)

(73) Assignee: THE BRIGHAM AND WOMEN'S HOSPITAL, INC., Boston, MA (US)

(21) Appl. No.: 15/770,535

(22) PCT Filed: Oct. 26, 2016
(86) PCT No.: PCT/US2016/058775

§ 371 Date: Apr. 24, 2018

Related U.S. Application Data
(60) Provisional application No. 62/246,388, filed on Oct. 26, 2015.

(51) Int. Cl. A61K 35/17; A61K 38/48; A61P 37/06

(52) U.S. Cl. CPC A61K 35/17; A61P 37/06; C12Y 304/22027; A61K 38/4873

(57) ABSTRACT

As described herein, the activity of Treg cells can be mediated by cathepsin inhibition, e.g., in tumor environments, cathepsin inhibition results in increased Treg anti-tumor activity, while in non-tumor environments, cathepsin inhibition results in increased immunosuppressive activity. Accordingly, provided herein are methods of modulating Treg activity and methods of treating diseases (e.g., cancer or autoimmune diseases) by inhibiting cathepsins in Treg cells.

### CROSS-REFERENCE TO RELATED APPLICATIONS

This application claims benefit under 35 U.S.C. § 119(e) of U.S. Provisional Application No. 62/246,388 filed Oct. 26, 2016, the contents of which are incorporated herein by reference in their entirety.

### GOVERNMENT SUPPORT

This invention was made with government support under Grant Nos. HL81090, HL60942, and HL123568 awarded by the National Institutes of Health. The U.S. government has certain rights in the invention.

### TECHNICAL FIELD

The technology described herein relates to increasing the level, activity and/or lifespan of regulatory T cells, and treatments for diseases related thereto.

### BACKGROUND

Autoimmune diseases can arise when the immune system becomes overactive. Instead of recognizing and destroying foreign entities, an unrestrained immune system will begin to attack a subject's own body. In a healthy subject, such diseases are prevented by regulatory T cells (Tregs), which act as a brake on the rest of the immune system, limiting its activity. If the Tregs themselves malfunction or prove insufficient to control the immune system, autoimmune diseases may arise. Accordingly, therapies have been developed for such conditions that attempt to increase the levels or activity of Tregs.

### SUMMARY

Existing therapies that target Tregs are only effective for very short periods of time. Tregs have a short lifespan after such treatments, and the subject must be continually re-treated in order to benefit from the treatment. As described herein, the inventors have discovered methods for engineering Tregs to display high levels of activity and strikingly long lifespan. Accordingly, provided herein are engineered Tregs as well as methods of making them and methods of treating disease.

### BRIEF DESCRIPTION OF THE DRAWINGS

FIGS. 1A-1D demonstrate that CatS deficiency or pharmacological inhibition decreases the expression of cleaved (active) TLR7 in the kidney. JPM labeling to detect active cathepsins (FIG. 1A); RT-PCR to determine TLR7 mRNA (FIG. 1B), and TLR7 immunoblot to detect TLR7 processing (FIG. 1C) in kidneys from different 24-week-old mice, as indicted. FIG. 1D depicts in vitro digestion and TLR7 immunoblot of full-length TLR7 immunoprecipitation purified from 24-week-old B6-Fas^{lpr}Ctss^{-/-} mouse kidney and incubated with human recombinant CatS for indicated times. Immunoblot in FIG. 1C is representative data. Number of mice per group is indicated in each bar. β-actin immunoblots were used for protein loading controls.

FIGS. 2A-2F demonstrate that CatS deficiency limits the expression of cleaved (active) TLR7 in Treg cells and suppresses TLR7 signaling. FIG. 2A depicts FACS analysis to determine the purity of magnetic bead-purified mouse splenic CD4⁺CD25⁺Foxp3⁺ Tregs. FIG. 2B depicts FACS to determine the purity of magnetic beads and cell sorter doubly purified Tregs. JPM labeling to detect active cathepsins (FIG. 2C), RT-PCR to determine TLR7 mRNA (FIG. 2D), and TLR7 immunoblot to determine cleaved TLR7 (FIG. 2E) in doubly purified spleen Treg cells from different 24-week-old mice, as indicted. FIG. 2F depicts immunoblots to detect p-MyD88 and p-NF-κB (p65) in doubly purified splenic Treg cells from different mice. Immunoblot in FIG. 2E is representative data. Number of mice per group is indicated in each bar. GAPDH or β-actin immunoblots were used for protein loading controls.

FIGS. 3A-3F demonstrate that CatS deficiency or pharmacological inhibition improves Treg biology in vitro and in B6-Fas^{lpr} mice. FIG. 3A depicts FACS analysis of CD4⁺CD25^{high}Foxp3⁺ Treg cells in splenic CD4⁺ T cells from different mice after stimulation with IL2 and TGF-β in the presence or absence of a CatS inhibitor. FIG. 3B demonstrates media IFN-γ levels from CD4⁺CD25⁻ Teff cells from B6-Fas^{lpr} mice after incubation for 2 days with or without anti-CD3 and anti-CD28 mAb and magnetic bead-purified Treg cells from different types of mice, as indicated. FIG. 3C depicts FACS that determined CD4⁺CD25^{high}Foxp3⁺ cell numbers per gram spleen. The top-to-bottom series listing below the graph corresponds to the series bars in left-to-right order. FIG. 3D demonstrates immunofluorescent staining determined CD4⁺Foxp3⁺ cells in kidney sections from B6-WT, B6-Fas^{lpr} mice, B6-Fas^{lpr} Ctss^{-/-} mice, and B6-Fas^{lpr} mice receiving different types of Treg cells. The top-to-bottom series listing below the graph corresponds to the series bars in left-to-right order. FIG. 3E depicts serum anti-histone, anti-ssDNA, anti-dsDNA, and RNP/Sm autoantibody titers from 24-week-old B6-WT, B6-Fas^{lpr}, B6-Fas^{lpr}Ctss^{-/-} mice, and B6-Fas^{lpr} mice receiving adoptive transfer of Treg cells from B6-WT and B6-Ctss^{-/-} mice or B6-WT Treg cells pre-treated with a CatS inhibitor. Number of mice per group is indicated in each parentheses. Repeated measures ANOVA. FIG. 3F depicts media IFN-γ levels from different Teff cells after 2 days of incubation with or without anti-CD3 and anti-CD28 mAb and magnetic beads combined with cell sorter-purified splenic Treg cells recovered from B6-Fas^{lpr} mice receiving different Treg cells as indicated. Data in FIGS. 3A, 3B, and 3F are representative of three independent experiments. P<0.05 is considered statistically significant, Mann-Whitney U test for FIGS. 3A-3D and 3F. Repeated measures ANOVA for FIG. 3E.

FIGS. 4A-4E demonstrate that CatS deficiency or inhibition enhances Treg function and their in-tissue survival and proliferation, and promotes autoantibody suppression in B6-Fas^{lpr} mice. All Treg cells were purified with magnetic beads followed by cell sorter. FIG. 4A depicts a graph of media IFN-γ levels from CD4⁺CD25⁻ Teff cells from B6-Fas^{lpr} mice after incubation with or without anti-CD3 and anti-CD28 mAb and different Treg cells. FIG. 4B depicts a graph of serum autoantibody titers from B6-Fas^{lpr} mice receiving differently treated Treg cells from CD45.1 mice. FIG. 4C depicts a graph of media IFN-γ levels from Teff cells from B6-Fas^{lpr} mice after incubation with or without anti-CD3 and anti-CD28 mAb and splenic Treg cells recovered from B6-Fas^{lpr} mice 15 weeks after adoptive transfer of different Treg cells. FIG. 4D depicts the results of FACS of CD45.1⁺Foxp3⁺ cells in splenocytes from B6-Fas^{lpr} recipient mice. Representative graphs are shown to the left. FIG. 4E depicts a graph of serum total and active forms of CatS in B6-Fas^{lpr} mice 10 weeks after receiving different Treg cells. Data are representative of three independent experiments in FIGS. 4A, 4C, and 4D. Repeated measures ANOVA (compared with the B6-Fas^{lpr} mice parental mice or those received untreated Treg cells) was used and number of mice per group is indicated in each parenthesis in FIGS. 4B and 4E.

FIGS. 5A-5B demonstrate that transfer of Treg cells into B6-Fas^{lpr} mice results in decreases TLR7 activation in the kidney. TLR7 immunoblot to detect cleaved TLR7 (FIG. 5A) and JPM labeling to detect active cathepsins (FIG. 5B) in kidneys from mice having received different types of Treg cells as indicated. Immunoblots in both are representative data. Number of mice per group is indicated in each bar. β-actin immunoblot was used for protein loading controls.

FIGS. 6A-6D demonstrate that CatS inhibition increases Treg activity in three human donors A, B, and C. FIG. 6A depicts the results of FACS which determined CD4⁺Foxp3⁺ Treg differentiation in human peripheral CD4⁺ T cells after 2 days of stimulation with or without IL2 and TGF-β in the presence or absence of 10 μg/mL CatS inhibitor. FIG. 6B depicts the results of FACS which determined CD4⁺Foxp3⁺ Treg differentiation in human peripheral CD4⁺ T cells in the presence or absence of a CatS inhibitor and stimulated with and without IL2 and TGF-β for indicated days. FIG. 6C depicts graphs of media IFN-γ and IL2 levels from human CD4⁺CD25⁻ Teff cells after 3 days of incubation in anti-CD3 and anti-CD28 mAb with and without untreated and CatS inhibitor overnight pre-treated human Treg cells from three donors, as indicated. FIG. 6D depicts a TLR7 immunoblot of human PBMCs from three donors treated overnight with and without a CatS inhibitor.

FIGS. 7A-7C demonstrate Treg analysis in spleen and tumor from C57BL/6 mice received without (Control, empty bars) or with CD45.1⁺ donor Tregs (CD45.1⁺ Treg, filled bars) at 7 days after MB49 tumor cell subcutaneous implantation. FIG. 7A demonstrates that FACS analysis detected CD4⁺CD25⁺ total Tregs in spleens and tumor tissues. Representative tumor cell FACS data are shown to the right panels. FIG. 7B demonstrates that FACS analysis detected CD45.1⁺Foxp3⁺ Tregs in spleens and tumor tissues. Representative tumor cell FACS data are shown to the right panels. Data are mean±SEM from six mice per group. P<0.05 was considered statistical significant, Mann-Whitney U test. FIG. 7C demonstrates that FITC-anti-mouse CD45.1 antibody-mediated immunofluorescent staining detected CD45.1⁺ cells in spleens and tumor tissues from mice received with (right panel) and without (left panel) CD45.1⁺ donor Tregs.

FIGS. 8A-8B depict FACS analysis of spleen and tumor CD4⁺CD25⁺Foxp3⁺ Tregs. FIG. 8A depicts Treg contents in splenocytes from mice received with and without PBS-treated or CatS inhibitor-treated Tregs. FIG. 8B depicts Treg contents in tumor single cell preparations from mice received with and without PBS-treated or CatS inhibitor-treated Tregs. Data are mean±SEM from 12 mice per group. Representative FACS data are shown to the right panels. P<0.05 was considered statistical significant, Mann-Whitney U test.

FIGS. 9A-9D depict spleen and tumor immunohistochemical analysis. FIG. 9A depicts spleen TUNEL-positive areas. FIG. 9B depicts tumor Ki67-positive areas. Representative data are shown to the right panels. FIG. 9C depicts spleen Ki67-positive areas. Representative data are shown to the right panels. FIG. 9D depicts tumor and spleen CD31-positive microvesel numbers per mm². Data are mean±SEM from 12 mice per group. P<0.05 was considered statistical significant, Mann-Whitney U test.

FIGS. 10A-10D demonstrate Treg and tumor cell proliferation and apoptosis in different co-cultures of Tregs, WT splenocytes, and MB49 tumor cells or tumor cell conditioned media. FIG. 10A depicts Treg proliferation (CD45.1⁺Ki67⁺). FIG. 10B depicts Treg apoptosis (CD45.1⁺Annexin V⁺). FIG. 10C depicts MB49 tumor cell proliferation (Ki67⁺). FIG. 10D depicts MB49 tumor cell apoptosis (Annexin V⁺). All different co-cultures are indicated. Data are mean±SEM from three independent experiments. P<0.05 was considered statistical significant, Mann-Whitney U test.

FIG. 11A-11F depict proliferation and apoptosis of B cells, CD4⁺ T cells, and CD8⁺ T cells in WT splenocytes after co-culture with different Tregs with and without MB49 tumor cell conditioned media. FIG. 11A depicts B-cell proliferation (CD45.1⁻B220⁺Ki67⁺). FIG. 11B depicts CD4⁺ T-cell proliferation (CD45.1⁻CD4⁺Ki67⁺). FIG. 11C depicts CD8⁺ T-cell proliferation (CD45.1⁻ CD8⁺Ki67⁺). FIG. 11D depicts B-cell apoptosis (CD45.1⁻B220⁺Annexin V⁺). FIG. 11E depicts CD4⁺ T-cell apoptosis (CD45.1⁻CD4⁺Annexin V⁺). FIG. 11F depicts CD8⁺ T-cell apoptosis (CD45.1⁻ CD8⁺Annexin V⁺). All different co-cultures are indicated. Data are mean±SEM from three independent experiments. P<0.05 was considered statistical significant, Mann-Whitney U test.

### DETAILED DESCRIPTION

Regulatory T cells (Tregs) are a specialized type of immune cell that control the overall immune response, for example, by suppressing the activity of other aspects of the immune system. Thus, Tregs normally act to downregulate the immune system, preventing overreactions that can result in autoimmune diseases. This natural role of Tregs can be exploited therapeutically, and a number of diseases can be treated by activating or stimulating a patient's Tregs. However, the existing methods of stimulating Tregs are only effective for exceptionally short periods of time, requiring constant redosing of the patient to provide a therapeutic effect.

Described herein is a method by which Tregs can be engineered to assume an activated status that persists for an execeptionally long time. Current methods of Treg activation result in Tregs that are active for periods of several days. The methods described herein provide Tregs that demonstrate activity over the course of several months.

In one aspect, described herein is a method of engineering a Treg cell, e.g., a Treg cell with a long-lived phenotype, the method comprising: contacting a Treg cell ex vivo with an inhibitor of cathepsin S, cathepsin K, and/or cathepsin L. In one aspect, described herein is a method of treating a Treg-mediated disease in a subject in need of treatment thereof, the method comprising: contacting a Treg cell ex vivo with an inhibitor of cathepsin S, cathepsin K, and/or cathepsin L; and administering the cell to the subject.

As used herein “cathepsin S” refers to a cysteine protease active at neutral pH which cleaves laminin, fibronectin elastin, osteocalcin, some collagens, chondroitin sulfate, heparan sulfate and proteoglycans of the basal membrane. Sequences for cathepsin S are known for a number of species, e.g., human cathepsin S (NCBI Gene ID: 1520). As used herein “cathepsin K” refers to a cysteine protease with a high specificity for kinins. Sequences for cathepsin K are known for a number of species, e.g., human cathepsin K (NCBI Gene ID: 1513). As used herein “cathepsin L” refers to a cysteine protease which cleaves collagen, elastin, and alpha-1 protease inhibitor. Sequences for cathepsin L are known for a number of species, e.g., human cathepsin L (NCBI Gene ID: 1514).

As used herein, the term “inhibitor” refers to an agent which can decrease the expression and/or activity of the targeted expression product, e.g. by at least 10% or more, e.g. by 10% or more, 50% or more, 70% or more, 80% or more, 90% or more, 95% or more, or 98% or more. The efficacy of an inhibitor of a particular target e.g. its ability to decrease the level and/or activity of the target can be determined, e.g. by measuring the level of an expression product and/or the activity of the target. Methods for measuring the level of a given mRNA and/or polypeptide are known to one of skill in the art, e.g. RT-PCR with primers can be used to determine the level of RNA and Western blotting with an antibody (e.g. an anti-cathepsin S antibody, e.g. Cat No. ab134157; Abcam; Cambridge, Mass.) can be used to determine the level of a polypeptide. The activity of a cathepsin can be determined using methods known in the art, e.g. measuring the level of one or more of its enzymatics targets. Changes in the molecular weights of one or more targets, indicating cleavage of the target, are readily detected by western blot. In some embodiments, the inhibitor can be an inhibitory nucleic acid; an aptamer; an antibody reagent; an antibody; or a small molecule.

In some embodiments, the inhibitor is an antibody reagent that binds specifically to cathepsin S, cathepsin K, and/or cathepsin L.

The inhibitors described herein can be inhibitors of cathepsin S, cathepsin K, and/or cathepsin L. In some embodiments, an inhibitor described herein can be a pan-cathepsin inhibitor, e.g. it can inhibit at least cathepsin S, K, and L and optionally, other cathepsin polypeptides. In some embodiments, an inhibitor described herein can inhibit cathepsin S, cathepsin K, and cathepsin L, but not other cathepsins. In some embodiments, an inhibitor described herein can inhibit cathepsin S and cathepsin K, but not other cathepsins. In some embodiments, an inhibitor described herein can be specific for cathepsin S, e.g., it inhibits only cathepsin S. In some embodiments, an inhibitor described herein can be specific for cathepsin K, e.g., it inhibits only cathepsin K. In some embodiments, an inhibitor described herein can be specific for cathepsin L, e.g., it inhibits only cathepsin L.

Inhibitors of cathepsins, including cathepsins S, K, and L are known in the art. Non-limiting examples of inhibitors specific for one or more of cathepsins S, K, and L can include, Fsn0503h humanize antibody; LY3000328; odancatib; balicatib; calpeptin; L006235; SID 26681509; VBY-891; VBY-129; VBY-825; and VBY-036. Further descriptions of cathepsin inhibitors and how to make them can be found, e.g., in U.S. Pat. Nos. 8,227,468; 8,975,296; 7,326,719; 8,877,967; 8,722,734; 8,293,722; and 8,680,152; US Patent Publications US2014/0155383; US2012/0329837; and US2006/0287402; International Patent Publications WO2000/049008; WO20012/156311; WO2005/019161; WO2005/040142; WO2012/151319; WO2014/164844; and WO2009/055467; EP Patent Publication 2635562; and Canadian Patent 2547591; each of which is incorporated by reference herein in its entirety.

In some embodiments, the methods described herein relate to contacting the cell with the inhibitor for a period of at least 2 hours. In some embodiments, the methods described herein relate to contacting the cell with the inhibitor for a period of at least 4 hours. In some embodiments, the methods described herein relate to contacting the cell with the inhibitor for a period of at least 6 hours. In some embodiments, the methods described herein relate to contacting the cell with the inhibitor for a period of no more than 24 hours. In some embodiments, the methods described herein relate to contacting the cell with the inhibitor for a period of no more than 12 hours. In some embodiments, the contact of the cell and the inhibitor can be stopped by, e.g., changing the media and/or isolating the cells from the media.

In some embodiments, the methods of treatment described herein can related to administering an autologous cell to the subject, e.g., a Treg cell can be isolated from the subject, engineered and/or contacted according to the methods described herein, and then administered to the subject.

As described elsewhere herein, the Treg cells described herein provide the surprising advantage of retaining their activity for a period of up to several months, as contrasted with current therapies where the cells lose their activity after a period of several days. Accordingly, the presently described methods and compositions permit much less frequent dosing of the subject. In some embodiments, the cells are administered no more frequently than once a month. In some embodiments, the cells are administered no more frequently than once every two months. In some embodiments, the cells are administered no more frequently than once every three months.

Additionally, because of the surprising efficacy of the cells and methods described herein, it is not necessary to treat the subject with compounds that activate Tregs. In some embodiments, the subject is not administered IL-2 or TGF-beta. In some embodiments, the subject is not administered an inhibitor of cathepsin S, cathepsin K, and/or cathepsin L.

In one aspect, described herein is a composition comprising a Treg cell and an inhibitor of cathepsin S, cathepsin K, and/or cathepsin L. In some embodiments, the inhibitor is present at a concentration sufficient to increase the activity, proliferation, and/or lifespan of the Treg cell. Methods of measuring Treg activity are described elsewhere herein. For example, Treg differentiation can be measured by detecting the expression of CD4 and Foxp3, or the immunosuppression of co-cultured Teff cells can be determined by measuring the level of IFN-gamma produced by the Teff cells. In some embodiments, the Treg cell is ex vivo and/or isolated. In some embodiments, the composition can further comprise, e.g. cell culture media.

Tregs which are engineered, e.g., treated and/or activated, according to the methods described herein are demonstrably different from naturally-occurring Tregs, as well as Tregs treated with, e.g., IL-2 or TGF-beta. For example, the Tregs described herein have a significantly longer lifespan and retain their activity for a markedly longer period of time. This difference in behavior is also reflected by structural differences that differentiate the presently described Tregs from previously-described (including naturally-occurring Tregs). In one aspect, described herein is an engineered Treg cell, the cell having a level of TLR7 polypeptide which is less than 50% of the level found in a naturally-occurring Treg cell, e.g., less than 50%, less than 40%, less than 30%, less than 20%, less than 10% or less. In one aspect, described herein is an engineered Treg cell, the cell having a level of activated TLR7 polypeptide which is less than 50% of the level found in a naturally-occurring Treg cell, e.g., less than 50%, less than 40%, less than 30%, less than 20%, less than 10% or less.

As used herein, “TLR7” or “Toll-like receptor 7” refers to a receptor protein that recognizes ssRNA in endosomes, e.g., recognition of viral infection. Sequences of TLR7 are known for a number of species, e.g., human TLR7 (NCBI Gene ID: 51284). Unactivated TRLR7 is present in a Treg as a polypeptide of about 125 kDa. TLR7 is activated by cleavage to form a polypeptide of about 70 kDa. In some embodiments, the level of activated TLR7 polypeptide is the level of the processed form of TLR7 having a molecular weight of about 70 kDa.

In some embodiments, cell has a level of unactivated TLR7 polypeptide which is 150% or greater than the level found in a naturally-occurring Treg cell, e.g. 150%, 200%, 300% or greater. In some embodiments, the level of unactivated TLR7 polypeptide is the level of TLR7 having a molecular weight of about 125 kDa.

Additional structural differences that distinguish the presently described Tregs can include increased levels of IL-2 (e.g., NCBI Gene ID: 3558) and TGF-beta (e.g., NCBI Gene ID: 7040) as well as decreased levels of IFN-γ (e.g., NCBI Gene ID: 3458) and IL-6 (e.g., NCBI Gene ID: 3569). In some embodiments, the cell described herein can further express a level of IFN-γ which is less than 50% of the level found in a naturally-occurring Treg cell (e.g., less than 50%, less than 40%, less than 30%, less than 20%, less than 10% or less); a level of IL-6 which is less than 50% of the level found in a naturally-occurring Treg cell (e.g., less than 50%, less than 40%, less than 30%, less than 20%, less than 10% or less); a level of IL-2 which is more than 150% of the level found in a naturally-occurring Treg cell (e.g. 150%, 200%, 300% or greater); and/or a level of TGF-β which is more than 150% of the level found in a naturally-occurring Treg cell (e.g. 150%, 200%, 300% or greater). In some embodiments, the cell described herein can further express a level of IFN-γ which is less than 50% of the level found in a naturally-occurring Treg cell (e.g., less than 50%, less than 40%, less than 30%, less than 20%, less than 10% or less); a level of IL-6 which is less than 50% of the level found in a naturally-occurring Treg cell (e.g., less than 50%, less than 40%, less than 30%, less than 20%, less than 10% or less); a level of IL-2 which is more than 150% of the level found in a naturally-occurring Treg cell (e.g. 150%, 200%, 300% or greater); and a level of TGF-β which is more than 150% of the level found in a naturally-occurring Treg cell (e.g. 150%, 200%, 300% or greater).

In one aspect, described herein is an engineered Treg cell, the cell expressing: a level of IFN-γ which is less than 50% of the level found in a naturally-occurring Treg cell; a level of IL-6 which is less than 50% of the level found in a naturally-occurring Treg cell; a level of IL-2 which is more than 150% of the level found in a naturally-occurring Treg cell; and/or a level of TGF-β which is more than 150% of the level found in a naturally-occurring Treg cell. In some embodiments, the cell expresses a level of IFN-γ which is less than 50% of the level found in a naturally-occurring Treg cell; a level of IL-6 which is less than 50% of the level found in a naturally-occurring Treg cell; a level of IL-2 which is more than 150% of the level found in a naturally-occurring Treg cell; and a level of TGF-β which is more than 150% of the level found in a naturally-occurring Treg cell.

Methods to measure gene expression products are known to a skilled artisan. Such methods to measure gene expression products, e.g., protein level, include ELISA (enzyme linked immunosorbent assay), western blot, immunoprecipitation, and immunofluorescence using detection reagents such as an antibody or protein binding agents.

In some embodiments, a cell described herein has been contacted with an inhibitor of cathepsin S, cathepsin K, and/or cathepsin L. In some embodiments, a cell described herein can be an isolated cell. In some embodiments, a cell described herein can be an ex vivo cell.

In some embodiments, the methods described herein relate to treating a subject having or diagnosed as having a Treg-mediated disease. A Treg-mediated disease can be any condition which is caused by, exacerbated by, and/or characterized by abnormally low levels of Tregs and/or Treg activity. Non-limiting examples of Treg-mediated diseases can include autoimmune disease; a cancer; a cardiovascular disease; a metabolic disease; systemic lupus erthythematosus; type I diabetes; arthritis; Sjoren's syndrome; type-II diabetes; obesity; atherosclerosis; abdominal aortic aneurysm; and/or transplant rejection (including organ transplantation, e.g., heart, liver, kidney, skin, lung, etc).

Subjects having a Treg disease, e.g., an autoimmune disease such as lupus can be identified by a physician using current methods of diagnosis. For example, symptoms and/or complications of lupus which characterize these conditions and aid in diagnosis are well known in the art and include but are not limited to, fever, joint pain, muscle pain, fatigue, low white blood cell counts, peripheral neuropathy, skin lesions anemia, etc. Tests that may aid in a diagnosis of, e.g. lupus include, but are not limited to, antinuclear antibody testing, anti-extractable nuclear antigen, complement level testing, kidney function testing, liver enzyme testing, and complete blood count. A family history of lupus, or exposure to risk factors for lupus can also aid in determining if a subject is likely to have lupus or in making a diagnosis of lupus.

The compositions and methods described herein can be administered to a subject having or diagnosed as having a Treg-mediated disease. In some embodiments, the methods described herein comprise administering an effective amount of compositions described herein, e.g. a Treg as described herein to a subject in order to alleviate a symptom of a Treg-mediated disease. As used herein, “alleviating a symptom” is ameliorating any condition or symptom associated with the disease. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount of a composition, e.g., a Treg as described herein, needed to alleviate at least one or more symptom of the disease or disorder, and relates to a sufficient amount of pharmacological composition to provide the desired effect. The term “therapeutically effective amount” therefore refers to an amount of a composition that is sufficient to provide a particular effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active ingredient, which achieves a half-maximal inhibition of symptoms) as determined in cell culture, or in an appropriate animal model. Levels in blood can be measured, for example, by high performance liquid chromatography. The effects of any particular dosage can be monitored by a suitable bioassay, e.g., assay for Treg activity, among others. The dosage can be determined by a physician and adjusted, as necessary, to suit observed effects of the treatment.

In some embodiments, the technology described herein relates to a pharmaceutical composition comprising a Treg as described herein, and optionally a pharmaceutically acceptable carrier. In some embodiments, the active ingredients of the pharmaceutical composition comprise a Treg as described herein. In some embodiments, the active ingredients of the pharmaceutical composition consist essentially of a Treg as described herein. In some embodiments, the active ingredients of the pharmaceutical composition consist of a Treg as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. a Treg as described herein.

In some embodiments, the pharmaceutical composition comprising a Treg as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of a Treg as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a composition as disclosed herein can also be incorporated into the parenteral dosage forms of the disclosure, including conventional and controlled-release parenteral dosage forms.

The methods described herein can further comprise administering a second agent and/or treatment to the subject, e.g. as part of a combinatorial therapy. By way of example, if the subject is a subject in need of treatment for cancer, non-limiting examples of a second agent and/or treatment can include radiation therapy, surgery, gemcitabine, cisplastin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DFMO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above. In addition, the methods of treatment can further include the use of radiation or radiation therapy. Further, the methods of treatment can further include the use of surgical treatments.

In certain embodiments, an effective dose of a composition comprising a Treg as described herein can be administered to a patient once. In certain embodiments, an effective dose of a composition comprising a Treg can be administered to a patient repeatedly. In some embodiments the dosage can be from about 1×10̂5 cells to about 1×10̂8 cells per kg of body weight. In some embodiments, the dosage can be from about 1×10̂6 cells to about 1×10̂7 cells per kg of body weight. In some embodiments, the dosage can be about 1×10̂6 cells per kg of body weight. In some embodiments, one dose of cells can be administered. In some embodiments, the dose of cells can be repeated, e.g., once, twice, or more.

In some embodiments, after an initial treatment regimen, the treatments can be administered on a less frequent basis. Treatment according to the methods described herein can reduce levels of a marker or symptom of a condition, by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the treatement. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising a Treg as described herein can be administered over a period of time, such as over a 5 minute, 10 minute, 15 minute, 20 minute, or 25 minute period.

The dosage ranges for the administration of a Treg as described herein according to the methods described herein depend upon, for example, the form of the composition, its potency, and the extent to which symptoms, markers, or indicators of a condition described herein are desired to be reduced, for example the percentage reduction desired for symptoms or the extent to which, for example, Treg activity is desired to be induced. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a Treg as described herein in, e.g. the treatment of a condition described herein, or to induce a response as described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. Treg activity. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of a mouse model of lupus. When using an experimental animal model, efficacy of treatment is evidenced when a statistically significant change in a marker is observed, e.g. Treg activity.

In vitro and animal model assays are provided herein which allow the assessment of a given dose of a Treg as described herein.

By way of non-limiting example, the effects of a dose of a Treg as described herein can be assessed by administering a Treg as described herein to a B6-Fas^{lpr} mouse and measuring the level of serum IL6 and IL17, where a decrease in IL6 and IL17 indicates an improvement in the condition of the mice. Alternatively, the level of autoantibody production can be measured.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a Treg-mediated disease, e.g. an autoimmune disease. A subject can be male or female.

A subject can be one who has been previously diagnosed with or identified as suffering from or having a condition in need of treatment (e.g. a Treg-mediated disease) or one or more complications related to such a condition, and optionally, have already undergone treatment for the disease or the one or more complications related to the disease. Alternatively, a subject can also be one who has not been previously diagnosed as having the disease or one or more complications related to the disease. For example, a subject can be one who exhibits one or more risk factors for the disease or one or more complications related to the disease or a subject who does not exhibit risk factors.

A “subject in need” of treatment for a particular condition can be a subject having that condition, diagnosed as having that condition, or at risk of developing that condition.

As used herein, “regulatory T cell” or “Treg” refers to T cells that suppress the function of other immune system cells.

As used herein, “engineered” refers to the aspect of having been manipulated by the hand of man. For example, a cell is considered to be “engineered” when at least one aspect of the cell has been manipulated by the hand of man to differ from the aspect as it exists in nature. As is common practice and is understood by those in the art, progeny an engineered cell are typically still referred to as “engineered” even though the actual manipulation was performed on a prior entity.

The term “isolated” or “partially purified” as used herein refers, in the case of a cell, to a cell separated from at least one other component that is present with cell as found in its natural source and/or that would be present with the cell when found in vitro. A cultured cell, or a cell existing ex vivo is considered “isolated.”

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In some embodiments, an inhibitor of a given polypeptide can be an antibody reagent specific for that polypeptide. As used herein an “antibody” refers to IgG, IgM, IgA, IgD or IgE molecules or antigen-specific antibody fragments thereof (including, but not limited to, a Fab, F(ab′)₂, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria.

As described herein, an “antigen” is a molecule that is bound by a binding site on an antibody agent. Typically, antigens are bound by antibody ligands and are capable of raising an antibody response in vivo. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

The terms “antigen-binding fragment” or “antigen-binding domain”, which are used interchangeably herein are used to refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

Additionally, and as described herein, a recombinant humanized antibody can be further optimized to decrease potential immunogenicity, while maintaining functional activity, for therapy in humans. In this regard, functional activity means a polypeptide capable of displaying one or more known functional activities associated with a recombinant antibody or antibody reagent thereof as described herein. Such functional activities include, e.g. the ability to bind to a target.

As used herein, “expression level” refers to the number of mRNA molecules and/or polypeptide molecules encoded by a given gene that are present in a cell or sample. Expression levels can be increased or decreased relative to a reference level.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

Inhibitors of the expression of a given gene can be an inhibitory nucleic acid. In some embodiments, the inhibitory nucleic acid is an inhibitory RNA (iRNA). As used herein, the term “iRNA” refers to any type of interfering RNA, including but are not limited to RNAi, siRNA, shRNA, endogenous microRNA and artificial microRNA. Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

As used herein, the term “iRNA” refers to an agent that contains RNA as that term is defined herein, and which mediates the targeted cleavage of an RNA transcript via an RNA-induced silencing complex (RISC) pathway. In one embodiment, an iRNA as described herein effects inhibition of the expression and/or activity of a target gene described herein. In certain embodiments, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iRNA.

In some embodiments, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions. Generally, the duplex structure is between 15 and 30 inclusive, more generally between 18 and 25 inclusive, yet more generally between 19 and 24 inclusive, and most generally between 19 and 21 base pairs in length, inclusive. Similarly, the region of complementarity to the target sequence is between 15 and 30 inclusive, more generally between 18 and 25 inclusive, yet more generally between 19 and 24 inclusive, and most generally between 19 and 21 nucleotides in length, inclusive. In some embodiments, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

In yet another embodiment, the RNA of an iRNA, e.g., a dsRNA, is chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In particular embodiments, the modified RNA will have a phosphorus atom in its internucleoside backbone.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, each of which is herein incorporated by reference

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other RNA mimetics suitable or contemplated for use in iRNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found, for example, in Nielsen et al., Science, 1991, 254, 1497-1500.

Some embodiments featured in the invention include RNAs with phosphorothioate backbones and oligonucleosides with heteroatom backbones, and in particular —CH₂—NH—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂— and —N(CH₃)—CH₂—CH₂—[wherein the native phosphodiester backbone is represented as —O—P—O—CH₂—] of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506.

Modified RNAs can also contain one or more substituted sugar moieties. The iRNAs, e.g., dsRNAs, featured herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Exemplary suitable modifications include O[(CH₂)ₙO]ₘCH₃, O(CH₂).ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples herein below.

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference.

An iRNA can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is herein incorporated by reference, and U.S. Pat. No. 5,750,692, also herein incorporated by reference.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193). Representative U.S. Patents that teach the preparation of locked nucleic acid nucleotides include, but are not limited to, the following: U.S. Pat. Nos. 6,268,490; 6,670,461; 6,794,499; 6,998,484; 7,053,207; 7,084,125; and 7,399,845, each of which is herein incorporated by reference in its entirety.

Another modification of the RNA of an iRNA as described herein involves chemically linking to the RNA one or more ligands, moieties or conjugates that enhance the activity, cellular distribution, pharmacokinetic properties, or cellular uptake of the iRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder associated. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method of treating a Treg-mediated disease in a subject in need of treatment thereof, the method comprising:
   a. contacting a Treg cell ex vivo with an inhibitor of cathepsin S, cathepsin K, and/or cathepsin L; and
   b. administering the cell to the subject
2. The method of paragraph 1, wherein the cell is autologous to the subject.
3. The method of any of paragraphs 1-2, wherein the Treg-mediated disease is an autoimmune disease; a cancer; a cardiovascular disease; or a metabolic disease.
4. The method of paragraph 3, wherein the autoimmune disease is selected from the group consisting of:
   systemic lupus erthythematosus; type I diabetes; arthritis; Sjoren's syndrome; type-II diabetes; obesity; atherosclerosis; abdominal aortic aneurysm; and transplant rejection. (heart, liver, kidney, skin, lung, etc).
5. The method of any of paragraphs 1-4, wherein the inhibitor is an inhibitor of cathepsin S.
6. The method of any of paragraphs 1-4, wherein the inhibitor is an inhibitor of cathepsin K.
7. The method of any of paragraphs 1-6, wherein the inhibitor is an inhibitor of cathepsin S and cathepsin K.
8. The method of any of paragraphs 1-4, wherein the inhibitor is an inhibitor of cathepsin L.
9. The method of any of paragraphs 1-8, wherein the inhibitor is an inhibitor of cathepsin S; cathepsin K; and cathepsin L.
10. The method of any of paragraphs 1-9, wherein the inhibitor is a small molecule selected from the group consisting of:
   LY3000328; odancatib; balicatib; calpeptin; L006235; SID 26681509; VBY-891; VBY-129; VBY-825; and VBY-036.
11. The method of any of paragraphs 1-9, wherein the inhibitor is an antibody reagent that binds specifically to cathepsin S, cathepsin K, and/or cathepsin L.
12. The method of any of paragraphs 1-11, wherein the cell is contacted with the inhibitor for a period of at least 6 hours.
13. The method of any of paragraphs 1-12, wherein the cell is contacted with the inhibitor for a period of no more than 24 hours.
14. The method of any of paragraphs 1-13, wherein the cell is contacted with the inhibitor for a period of no more than 12 hours.
15. The method of any of paragraphs 1-14, wherein the cells are administered no more frequently than once a month.
16. The method of any of paragraphs 1-15, wherein the cells are administered no more frequently than once every two months.
17. The method of any of paragraphs 1-16, wherein the cells are administered no more frequently than once every three months.
18. The method of any of paragraph 1-17, wherein the subject is not administered an inhibitor of cathepsin S, cathepsin K, and/or cathepsin L.
19. The method of any of paragraphs 1-18, wherein the subject is not administered IL-2 or TGF-beta.
20. A composition comprising a Treg cell and an inhibitor of cathepsin S, cathepsin K, and/or cathepsin L.
21. The composition of paragraph 20, wherein the inhibitor is present at a concentration sufficient to increase the activity, proliferation, and/or lifespan of the Treg cell.
22. An engineered Treg cell, the cell having a level of TLR7 polypeptide which is less than 50% of the level found in a naturally-occurring Treg cell.
23. An engineered Treg cell, the cell having a level of activated TLR7 polypeptide which is less than 50% of the level found in a naturally-occurring Treg cell.
24. The cell of paragraph 23, wherein the activated TLR7 polypeptide is the processed form of TLR7 having a molecular weight of about 70 kDa.
25. The cell of any of paragraphs 22-24, wherein the level of TLR7 polypeptide is less than 20% of the level found in a naturally-occurring Treg cell.
26. The cell of any of paragraphs 22-25, wherein the level of TLR7 polypeptide is less than 10% of the level found in a naturally-occurring Treg cell.
27. The cell of any of paragraphs 22-26, wherein the cell has a level of unactivated TLR7 polypeptide which is 150% or greater than the level found in a naturally-occurring Treg cell.
28. The cell of paragraph 27, wherein the unactivated TLR7 polypeptide is the form of TLR7 having a molecular weight of about 125 kDa.
29. The cell of any of paragraphs 22-28, wherein the cell expresses:
   a level of IFN-γ which is less than 50% of the level found in a naturally-occurring Treg cell;
   a level of IL-6 which is less than 50% of the level found in a naturally-occurring Treg cell;
   a level of IL-2 which is more than 150% of the level found in a naturally-occurring Treg cell; and/or
   a level of TGF-β which is more than 150% of the level found in a naturally-occurring Treg cell.
30. An engineered Treg cell, the cell expressing:
   a level of IFN-γ which is less than 50% of the level found in a naturally-occurring Treg cell;
   a level of IL-6 which is less than 50% of the level found in a naturally-occurring Treg cell;
   a level of IL-2 which is more than 150% of the level found in a naturally-occurring Treg cell; and/or
   a level of TGF-β which is more than 150% of the level found in a naturally-occurring Treg cell.
31. The cell of paragraph 30, wherein the cell expresses:
   a level of IFN-γ which is less than 50% of the level found in a naturally-occurring Treg cell;
   a level of IL-6 which is less than 50% of the level found in a naturally-occurring Treg cell;
   a level of IL-2 which is more than 150% of the level found in a naturally-occurring Treg cell; and
   a level of TGF-β which is more than 150% of the level found in a naturally-occurring Treg cell.
32. The cell of any of paragraphs 22-31, wherein the cell has been contacted with an inhibitor of cathepsin S, cathepsin K, and/or cathepsin L.
33. The cell of any of paragraphs 22-32, wherein the cell is an isolated cell.

Some embodiments of the technology described herein can be defined according to any of the following numbered paragraphs:
1. A method of treating a Treg-mediated disease in a subject in need of treatment thereof, the method comprising:
   a. contacting a Treg cell ex vivo with an inhibitor of cathepsin S, cathepsin K, and/or cathepsin L; and
   b. administering the cell to the subject
2. The method of paragraph 1, wherein the cell is autologous to the subject.
3. The method of any of paragraphs 1-2, wherein the Treg-mediated disease is an autoimmune disease; a cancer; a cardiovascular disease; or a metabolic disease.
4. The method of paragraph 3, wherein the autoimmune disease is selected from the group consisting of:
   systemic lupus erthythematosus; type I diabetes; arthritis; Sjoren's syndrome; type-II diabetes; obesity; atherosclerosis; abdominal aortic aneurysm; and transplant rejection. (heart, liver, kidney, skin, lung, etc).
5. The method of any of paragraphs 1-4, wherein the inhibitor is an inhibitor of cathepsin S.
6. The method of any of paragraphs 1-4, wherein the inhibitor is an inhibitor of cathepsin K.
7. The method of any of paragraphs 1-6, wherein the inhibitor is an inhibitor of cathepsin S and cathepsin K.
8. The method of any of paragraphs 1-4, wherein the inhibitor is an inhibitor of cathepsin L.
9. The method of any of paragraphs 1-8, wherein the inhibitor is an inhibitor of cathepsin S; cathepsin K; and cathepsin L.
10. The method of any of paragraphs 1-9, wherein the inhibitor is a small molecule selected from the group consisting of:
   LY3000328; odancatib; balicatib; calpeptin; L006235; SID 26681509; VBY-891; VBY-129; VBY-825; and VBY-036.
11. The method of any of paragraphs 1-9, wherein the inhibitor is an antibody reagent that binds specifically to cathepsin S, cathepsin K, and/or cathepsin L.
12. The method of any of paragraphs 1-11, wherein the cell is contacted with the inhibitor for a period of at least 6 hours.
13. The method of any of paragraphs 1-12, wherein the cell is contacted with the inhibitor for a period of no more than 24 hours.
14. The method of any of paragraphs 1-13, wherein the cell is contacted with the inhibitor for a period of no more than 12 hours.
15. The method of any of paragraphs 1-14, wherein the cells are administered no more frequently than once a month.
16. The method of any of paragraphs 1-15, wherein the cells are administered no more frequently than once every two months.
17. The method of any of paragraphs 1-16, wherein the cells are administered no more frequently than once every three months.
18. The method of any of paragraph 1-17, wherein the subject is not administered an inhibitor of cathepsin S, cathepsin K, and/or cathepsin L.
19. The method of any of paragraphs 1-18, wherein the subject is not administered IL-2 or TGF-beta.
20. The method of any of paragraphs 1-19, wherein the patient has both a) an autoimmune disease; a cardiovascular disease; or a metabolic disease; and b) a cancer.
21. A composition comprising a Treg cell and at least one inhibitor of cathepsin S, cathepsin K, and/or cathepsin L.
22. The composition of paragraph 21, wherein the inhibitor is present at a concentration sufficient to increase the activity, proliferation, and/or lifespan of the Treg cell.
23. An engineered Treg cell, the cell having a level of TLR7 polypeptide which is less than 50% of the level found in a naturally-occurring Treg cell.
24. An engineered Treg cell, the cell having a level of activated TLR7 polypeptide which is less than 50% of the level found in a naturally-occurring Treg cell.
25. The cell of paragraph 24, wherein the activated TLR7 polypeptide is the processed form of TLR7 having a molecular weight of about 70 kDa.
26. The cell of any of paragraphs 23-25, wherein the level of TLR7 polypeptide is less than 20% of the level found in a naturally-occurring Treg cell.
27. The cell of any of paragraphs 23-26, wherein the level of TLR7 polypeptide is less than 10% of the level found in a naturally-occurring Treg cell.
28. The cell of any of paragraphs 23-27, wherein the cell has a level of unactivated TLR7 polypeptide which is 150% or greater than the level found in a naturally-occurring Treg cell.
29. The cell of paragraph 28, wherein the unactivated TLR7 polypeptide is the form of TLR7 having a molecular weight of about 125 kDa.
30. The cell of any of paragraphs 23-29, wherein the cell expresses:
   a level of IFN-γ which is less than 50% of the level found in a naturally-occurring Treg cell;
   a level of IL-6 which is less than 50% of the level found in a naturally-occurring Treg cell;
   a level of IL-2 which is more than 150% of the level found in a naturally-occurring Treg cell; and/or
   a level of TGF-β which is more than 150% of the level found in a naturally-occurring Treg cell.
31. An engineered Treg cell, the cell expressing:
   a level of IFN-γ which is less than 50% of the level found in a naturally-occurring Treg cell;
   a level of IL-6 which is less than 50% of the level found in a naturally-occurring Treg cell;
   a level of IL-2 which is more than 150% of the level found in a naturally-occurring Treg cell; and/or
   a level of TGF-β which is more than 150% of the level found in a naturally-occurring Treg cell.
32. The cell of paragraph 31, wherein the cell expresses:
   a level of IFN-γ which is less than 50% of the level found in a naturally-occurring Treg cell;
   a level of IL-6 which is less than 50% of the level found in a naturally-occurring Treg cell;
   a level of IL-2 which is more than 150% of the level found in a naturally-occurring Treg cell; and
   a level of TGF-β which is more than 150% of the level found in a naturally-occurring Treg cell.
33. The cell of any of paragraphs 23-32, wherein the cell has been contacted with an inhibitor of cathepsin S, cathepsin K, and/or cathepsin L.
34. The cell of any of paragraphs 23-33, wherein the cell is an isolated cell.
35. A composition comprising a Treg cell contacted ex vivo with an inhibitor of cathepsin S, cathepsin K, and/or cathepsin L for the treatment of a Treg-mediated disease.
36. The composition of paragraph 35, wherein the cell is autologous to the subject.
37. The composition of any of paragraphs 35-36, wherein the Treg-mediated disease is an autoimmune disease; a cancer; a cardiovascular disease; or a metabolic disease.
38. The composition of paragraph 37, wherein the autoimmune disease is selected from the group consisting of:
   systemic lupus erthythematosus; type I diabetes; arthritis; Sjoren's syndrome; type-II diabetes; obesity; atherosclerosis; abdominal aortic aneurysm; and transplant rejection. (heart, liver, kidney, skin, lung, etc).
39. The composition of any of paragraphs 35-38, wherein the inhibitor is an inhibitor of cathepsin S.
40. The composition of any of paragraphs 35-39, wherein the inhibitor is an inhibitor of cathepsin K.
41. The composition of any of paragraphs 35-40, wherein the inhibitor is an inhibitor of cathepsin S and cathepsin K.
42. The composition of any of paragraphs 35-41, wherein the inhibitor is an inhibitor of cathepsin L.
43. The composition of any of paragraphs 35-42, wherein the inhibitor is an inhibitor of cathepsin S; cathepsin K; and cathepsin L.
44. The composition of any of paragraphs 35-43, wherein the inhibitor is a small molecule selected from the group consisting of:
   LY3000328; odancatib; balicatib; calpeptin; L006235; SID 26681509; VBY-891; VBY-129; VBY-825; and VBY-036.
45. The composition of any of paragraphs 35-44, wherein the inhibitor is an antibody reagent that binds specifically to cathepsin S, cathepsin K, and/or cathepsin L.
46. The composition of any of paragraphs 35-45, wherein the cell is contacted with the inhibitor for a period of at least 6 hours.
47. The composition of any of paragraphs 35-46, wherein the cell is contacted with the inhibitor for a period of no more than 24 hours.
48. The composition of any of paragraphs 35-47, wherein the cell is contacted with the inhibitor for a period of no more than 12 hours.
49. The composition of any of paragraphs 35-48, wherein the cells are administered no more frequently than once a month.
50. The composition of any of paragraphs 35-49, wherein the cells are administered no more frequently than once every two months.
51. The composition of any of paragraphs 35-50, wherein the cells are administered no more frequently than once every three months.
52. The composition of any of paragraphs 35-51, wherein the subject is not administered an inhibitor of cathepsin S, cathepsin K, and/or cathepsin L.
53. The composition of any of paragraphs 35-52, wherein the subject is not administered IL-2 or TGF-beta.
54. The composition of any of paragraphs 35-53, wherein the patient receiving the treatment has both a) an autoimmune disease; a cardiovascular disease; or a metabolic disease; and b) a cancer.

### EXAMPLES

### Example 1: Cathepsin S Suppresses Regulatory T Cells in Mice and Humans by Activating Toll-Like Receptor-7

Methods.

Using the lupus-prone Fas^{lpr} mice and human peripheral blood mononuclear cells, the role of CatS in mouse and human regulatory T-cell (Treg) toll-like receptor 7 (TLR7) expression and activation, Treg survival, immunosuppression, proliferation, and lupus mitigation was investigated.

Results.

CatS increases the expression of the active form of TLR7 in immune cells in mice and humans and in murine kidneys and enhances MyD88 and NF-κB activation. CatS deficiency or pharmacological inhibition enhanced splenic CD4⁺CD25^{high}Foxp3⁺ Treg cell differentiate, their lifespan and T effector cell immunosuppressive function. Transfer of in vitro prepared Treg cells into lupus-prone B6-Fas^{lpr} mice reduced serum autoantibody titers and more interestingly, transfer of Treg cells isolated from CatS-deficient mice or from normal mice and treated overnight with a CatS inhibitor reduced further serum autoantibody levels. Also, such transferred Treg cells could be found at increased numbers fifteen weeks later in the spleens and kidneys of B6-Fas^{lpr} mice with sustained high immunosuppressive activity.

Conclusion.

CatS is involved in the propagation of the autoimmune response through a previously unidentified mechanism, which involves the activation of TLR7 and the suppression of Treg cell function. This new H-2-independent mechanism indicates the use of CatS inhibitor-treated Treg cells to treat patients with systemic autoimmunity who are MHC polymorphic.

Introduction.

Cathepsin S (CatS) mediates invariant chain (CD74) stepwise proteolysis in antigen presenting cells (APCs) and is important in major histocompatibility complex (MHC) class-II antigenic peptide loading and antigen presentation (1, 2).

It has been shown that CatS activates Toll-like receptors (TLRs) TLR7 and TLR9 (11, 12) which bind viral nucleic acids and activate the NF-κB and interferon (IFN) regulatory factor (IRF) signaling pathways, thereby triggering the production of inflammatory cytokines (e.g., IL6) and type-1 IFNs (e.g., IFN-α and IFN-β), respectively (13). Genetic absence of TLR7 diminishes autoantibodies against Smith antigen (Sm) or ribonucleoprotein (RNP)-Sm complex (RNP/Sm) and reduces kidney glomerular IgG and C3 deposition and glomerulonephritis (14), whereas TLR9 function in lupus remains controversial (14, 15). Patients with systemic lupus erythematosus (SLE) have increased IL6, IL17, and IFN-α levels which correlate with disease activity and manifestations (16). These cytokines interfere or diminish regulatory T-cell (Treg) suppression of CD4⁺ T-effector cells (Teff) (12, 17), leading to Teff resistance among SLE patients. Lastly, genetic duplication of TLR7 leads to lupus-like disease and autoimmunity in humans and the BXSB mice (18-20).

It was investigated whether CatS activates TLR7 and interferes with Treg function. Such an effect would explain why its inhibition mitigates lupus-related manifestations in both H-2^{b} and H-2^{k} mice. Indeed, it is demonstrated herein that CatS inhibition in mice and humans promotes Treg function and at least in mice transfer of Treg cells from CatS-deficient or those pre-treated with a CatS inhibitor mitigates lupus-like autoimmunity in the B6-Fas^{lpr} mice.

Materials and Methods

Mice.

B6-Fas^{lpr} (N11), B6-Tlr7^{-/-} (N10), MRL/MpJ-Fas^{lpr}, and B6-CD45.1 transgenic mice (N25) were purchased from the Jackson Laboratory. B6-Tlr9^{-/-} mice were purchased from the Mutant Mouse Regional Resource Centers at the Jackson Laboratory. B6-Ctss^{-/-} mice (N15) were described previously (3). B6-Fas^{lpr} mice were crossbred with B6-Ctss^{-/-} mice to generate B6-Fas^{lpr} Ctss^{-/-} mice.

To perform Treg adoptive transfer in B6-Fas^{lpr} mice, spleen CD4⁺CD25⁺ Treg cells were purified from B6-WT, B6-Ctss^{-/-}, or B6-CD45.1 mice according to the manufacturer's instructions (Miltenyi Biotec, Inc., Auburn, Calif.). The resulting CD45.1⁺CD4⁺CD25⁺ Treg cells were also further purified with cell sorter (The BD FACSAira™ Cell Sorter, BD Biosciences, San Jose, Calif.). Treg purity was confirmed by FACS and anti-Foxp3 antibody-mediated immunofluorescent staining. WT Treg cells were incubated with a CatS inhibitor (10 μg/mL, clinicaltrials.gov/show/NCT01515358) overnight before adoptive transfer. Each 9-week-old female B6-Fas^{lpr} recipient mouse received intravenous injection of 5×10⁶ donor Treg cells. Blood samples and urine samples were collected biweekly three weeks after the adoptive transfer for 12 weeks. At the age of 24 weeks, mice were sacrificed and splenocytes were analyzed for CD4, CD25, and Foxp3 by FACS and kidneys were collected for CD4⁺Foxp3⁺ Treg immunofluorescent staining.

Immunofluorescent Staining.

Frozen kidney and spleen sections (5 μm), splenocytes, and purified Tregs were prepared for immunofluorescent staining using anti-CD45.1 (1:100, BioLegend, San Diego, Calif.), Ki67 (1:100, BioLegend), CD4 (1:250, Abnova, Walnut Calif.), and Foxp3 (1:100, eBioscience, San Diego, Calif.) monoclonal antibodies (mAb).

ELISA.

Serum autoantibodies were assessed by ELISA as described (21). NUNC maxisorp ELISA plates were pre-coated with ssDNA (100 μg/ml), dsDNA (100 μg/ml), histone (20 μg/ml) and RNP/Sm (20 μg/ml) in PBS at 4° C. overnight. Plates were blocked with 3% FCS for 1 h at 37° C., washed, and incubated with 1/300˜1/1000 dilutions of mouse sera for 1 h at 37° C. Anti-ssDNA antibody (clone TNT-3, IgG2a, Abcam, Cambridge, Mass.), anti-dsDNA antibody (clone HYB331-01, IgG2a, Abcam), anti-histones (clone 2Q2205, IgG2a, Abcam), and anti-RNP/Sm antibody (clone NB600-546, IgG3 Kappa, NOVUS Biologicals, Littleton, Colo.) were used as standards. Plates were washed, and a 1/1000 dilution of alkaline phosphatase-linked corresponding goat anti-mouse IgG2a or IgG3 secondary antibodies (Santa Cruz Biotechnology Inc., Santa Cruz, Calif.) in PBS were added for 1 hour at 37° C., and developed with a phosphatase substrate for 30 minutes at 37° C. Mouse serum total CatS, pro-CatS levels were determined according to the manufacturer's instructions, including total CatS DuoSet (DY1183, R&D systems, Minneapolis, Minn.), pro-CatS DuoSet (DY2227, R&D systems). Active CatS levels were determined by subtracting Pro-CatS from total CatS.

Antibodies and Flow Cytometry Analysis.

The following antibodies were used for FACS analysis: FcR-blocking antibody anti-CD16/32 (eBioscience), anti-CD4-Alexa Fluor 488, anti-CD25-PE, anti-mFoxp3-Alexa Fluor 647, anti-mouse-PE and all isotype controls (all from BD Biosciences). To determine the proportion of CD4⁺CD25^{high}Foxp3⁺ Treg cells in splenocytes, 100 μl of splenocyte suspension (˜1×10⁷ cell) was incubated at 4° C. in a phosphate buffered saline containing 2% fetal calf serum (FCS) with the Alexa Fluor 488-conjugated anti-CD4 and PE-conjugated anti-CD25 fluorescent antibodies followed by Alexa Fluor 647-conjugated FoxP3 intracellular staining. Staining for intracellular Foxp3 was performed using the fixation/permeabilization solution kit (BD Biosciences). Isotype controls were used for each antibody. Cells were acquired and analyzed with a FACSCalibur™ flow cytometer using CellQuest™ research software (version 3.3, BD Biosciences).

Real-Time PCR, Western Blot Analysis, and JPM Labeling.

Total RNA was prepared from kidney tissue or Treg cells using the Qiagen mini kit (Qiagen Inc., Valencia, Calif.). RNA concentration and quality were evaluated using the Agilent 2100™ bioanalyzer (Nano LabChip, Agilent Technologies, Santa Clara Calif.). After the cDNA synthesis, gene expression was quantified by real-time PCR on the ABI Prism 7900™ sequence detection system (Taqman, Applied Biosystems Inc., Foster City, Calif.). The low-density array detected 5 genes in one run in triplicates including endogenous controls (β-actin) and the mRNA levels of TLR7.

For immunoblot analysis, an equal amount of protein from each cell type or kidney lysate preparation was separated on a SDS-PAGE, blotted, and detected with different antibodies, including TLR7 (1:500, Abcam), phosphor-MyD88 (1:500, Cell Signaling Technology, Danvers, Mass.), phosphor-p65 NF-κB (1:500, Cell Signaling Technology), β-actin (1:3,000, Santa Cruz Biotechnology Inc.), and glyceraldehyde 3-phosphate dehydrogenase (GAPDH, 1:2000, Cell Signaling Technology).

The JPM probe was used to label and detect active cathepsins in kidney tissue extracts or Treg cell lysate. Tissues or cells were lysed in a pH 5.5 lysis buffer containing 1% Triton X-100, 40 mM sodium acetate, and 1 mM EDTA. Cathepsin active site JPM labeling was performed according to our prior studies (9, 22).

Teff and Treg Cell Preparation from Spleen Cells.

Mouse CD4⁺CD25⁻ Teff cells and CD4⁺CD25^{-/-} Treg cells in this study were purified from splenocytes. Briefly, splenocytes were collected from 6˜10-week-old mice or 24-week-old B6-Fas^{lpr} mice that received adoptive transfer of in vitro prepared Treg cells. Single cell suspensions were incubated with biotinylated-antibody cocktail containing antibodies against CD8a, CD11b, CD45R, CD49b and Ter-119 to deplete macrophages, granulocytes, B cells, and CD8⁺ T cells by negative selection. CD25⁺ cells were isolated from the CD4⁺ cell population by staining with PE-conjugated anti-CD25 antibody followed by incubation with magnetic-activated cell sorting (MACS) anti-PE microbeads (Miltenyi Biotec, Inc.). CD4⁺CD25^{-/-} T cells were then positively selected on a MACS mini-separation magnetic column, and the flow through fraction containing CD4⁺CD25⁻ T cells were collected. More than 90% of these cells were Treg cells that are positive for CD4 and CD25, as confirmed by FACS. Negative selection of CD25⁺ cells yielded CD4⁺CD25⁻ Teff cells. In some experiments, we performed a second round of CD4⁺CD25⁺ Treg purification using a cell sorter with magnetic bead-purified Treg cells as starting material.

Treg Activity Assay in Suppressing Teff Cells.

To test the function of Treg cells from B6-WT, B6-Fas^{lpr}, B6-Ctss^{-/-}, and B6-Fas^{lpr}Ctss^{-/-} mice and those from B6-Fas^{lpr} recipient mice that received donor Treg cells, a CD4⁺CD25⁺ Treg isolation kit with or without a second round of further purification with a cell sorter was used to isolate Treg cells. CD4⁺CD25⁻ Teff cells (3×10⁴ cells) were used as responder T cells and co-cultured with or without CD4⁺CD25⁺ Treg cells (3×10⁴ cells) with or without anti-CD3 mAb (2 μg/mL, clone OKT3, eBioscience) and anti-CD28 mAb (2.5 μg/ml, clone L293, BD Biosciences) or Treg cells pre-treated overnight with a CatS inhibitor (10 μg/mL) (23). Co-cultures were maintained in complete RPMI 1640 medium for 2 days. Culture media were collected for ELISA (eBioscience) to determine IFN-γ. All experiments were performed in triplicates.

In Vitro Treg Differentiations.

Naïve spleen cells or naïve CD4⁺ T cells purified from spleen cells (Miltenyi Biotec, Inc.) were cultured on an anti-CD3 mAb-pre-coated (5 μg/mL, eBioscience) 96-well plate in 200 mL complete RPMI 1640 containing 5 μg/mL anti-CD28 mAb (eBioscience) and stimulated with or without TGF-β (5 ng/mL, R&D Systems) and IL2 (200 U/mL, R&D System) for 2 days. Splenocytes and CD4⁺ T cells were collected and analyzed for CD4 and Foxp3 expression by FACS.

CatS Activity in Human Peripheral Blood Mononuclear Cells (PBMC), TLR7 Expression, and Treg Differentiation, Survival, and Immunosuppression.

Human PBMCs were purified from whole blood from three normal individuals from the blood bank of the Massachusetts General Hospital. Partners Human Research Committee approved the use of unidentified human blood specimens (protocol #2010-P-001930/2). PBMCs were incubated with and without a CatS inhibitor (10 μg/mL) overnight and their lysates were subjected to immunoblot analysis to detect both full-length and processed human TLR7. To purify human Treg and Teff cells, we first depleted non-CD4⁺ T cells using a cocktail of biotin-conjugated antibodies against CD8, CD14, CD15, CD16, CD19, CD36, CD56, CD123, TCRγ/δ and CD235a (glycophorin A), followed by anti-biotin MicroBeads separation. CD4⁺CD25⁺ Treg and CD4⁺CD25⁻ Teff cells were separated from purified CD4⁺ T cells using the CD25 MicroBeads for subsequent positive selection, according to the manufacturer (Miltenyi Biotec, Inc.). Secondary purification was performed using the cell sorter and the magnetic bead-purified Treg cells as starting material.

Purified CD4⁺ T cells from human PBMCs (Miltenyi Biotec, Inc.) were cultured on an anti-CD3 mAb-pre-coated (5 μg/mL, eBioscience) 96-well plate in 200 μL complete RPMI 1640 containing 5 μg/mL anti-CD28 mAb (eBioscience) and stimulated with or without TGF-β (5 ng/mL, R&D Systems), IL2 (200 U/mL, R&D System), and CatS inhibitor (10 ug/mL) for 1, 2, or 5 days, or additional 5 days without treatment. CD4⁺ T cells were collected and analyzed for CD4, CD25, and Foxp3 expression by FACS.

To test the function of human Treg cells, we incubated CD4⁺CD25⁻ Teff cells (1×10⁴) with or without purified human CD4⁺CD25^{high} Treg cells (1×10⁴) pre-treated with and without CatS inhibitor (10 μg/mL) overnight in the presence of anti-CD3 (2 μg/mL, eBioscience) and anti-CD28 (2.5 μg/mL, BD Biosciences) mAbs. All incubations were run in triplicates in 96-well plates with a final volume of 200 μl. At 3 days, supernatants were evaluated for IFN-γ and IL2 production by ELISA (BD Pharmingen).

Statistical Analysis.

Data were analyzed using non-parametric Mann-Whitney U test followed by Bonferroni corrections due to small sample sizes and often skewed data distributions. To simplify the data presentation of autoantibodies and serum CatS levels, we did not compare data from each time point, but rather compared all time point together as a whole from each group of mice using repeated-measures ANOVA. All data are presented as mean±SEM. P<0.05 is considered statistically significant.

Results

CatS Mediates TLR7 Expression and Activation in the Kidney and Treg Cells.

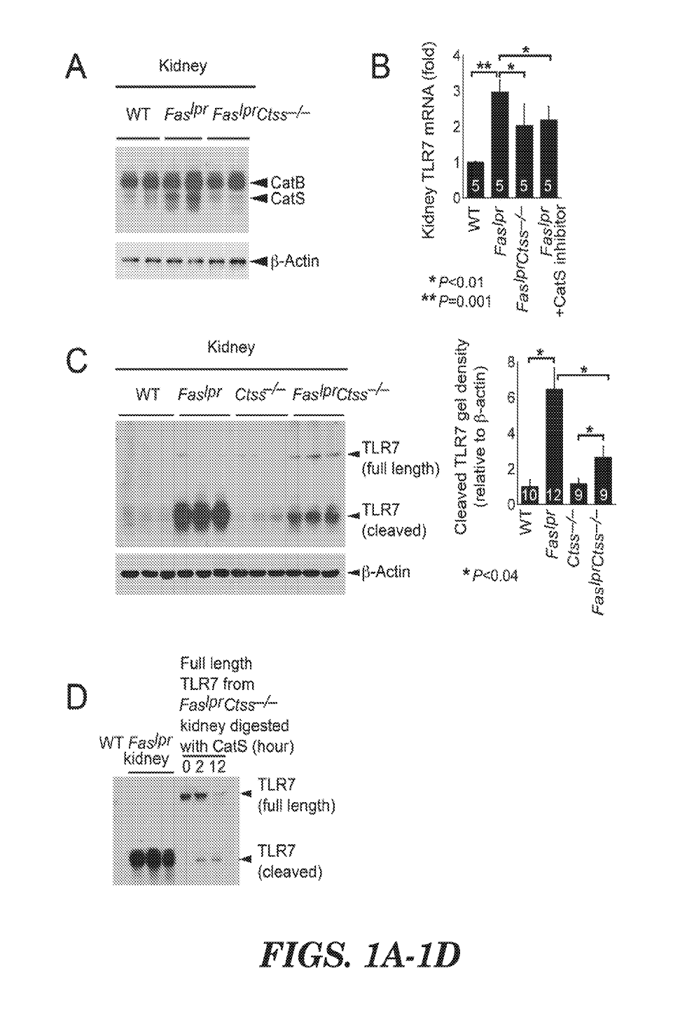

B6-Fas^{lpr} mice develop spontaneous SLE-like manifestations after 13-14 weeks with lupus nephritis in a manner similar to that observed in patients with SLE (14). JPM probe (targeting cysteine protease active site) labeled kidney tissue lysates from 24-week-old B6-Fas^{lpr} mice and revealed elevated CatS activity (3, 22). CatS activity disappeared in kidney extracts from CatS-deficient B6-Fas^{lpr}Ctss^{-/-} mice (FIG. 1A). TLR7 expression has been known to be increased in lupus nephritic kidneys from B6-Fas^{lpr} mice (24) and CatS deficiency reduced TLR7 mRNA levels (FIG. 1B). Next kidney lysates were subjected to immunoblot analysis with an anti-TLR7 antibody and it was found that B6-Fas^{lpr} kidneys displayed an accumulation of the 72-kDa cleaved (active) TLR7 fragment, whereas kidney lysates from B6-Fas^{lpr} Ctss^{-/-} mice showed accumulation of the 120-kDa full-length (inactive) TLR7 and reduction of the cleaved TLR7 fragment (FIG. 1C). To test directly whether CatS cleaves TLR7, the 120-kDa full-length TLR7 was purified from the B6-Fas^{lpr}Ctss^{-/-} mouse renal protein extracts, digested with a recombinant human CatS, and the time-dependent production of the 72-kDa cleaved TLR7 (11, 12) was determined. The presence of purified CatS resulted in disappearance of the full length and the appearance of the cleaved TLR7 (FIG. 1D).

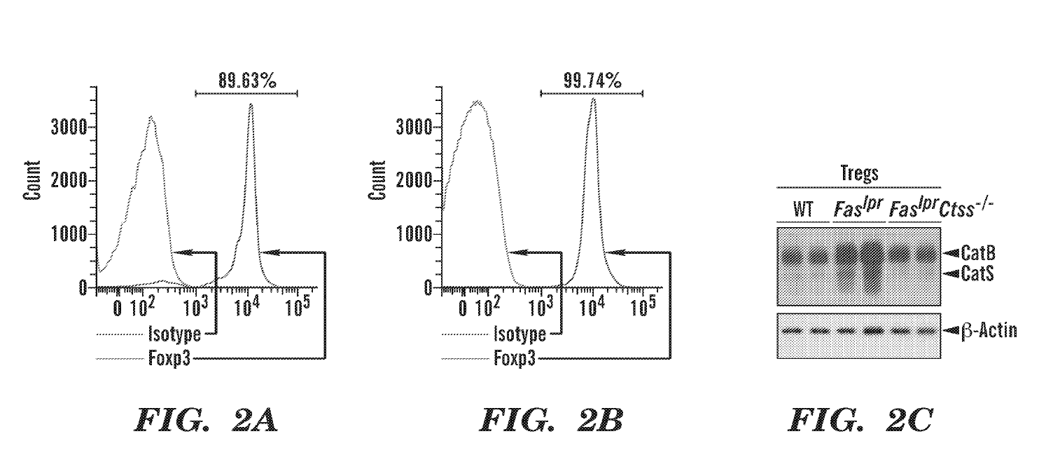

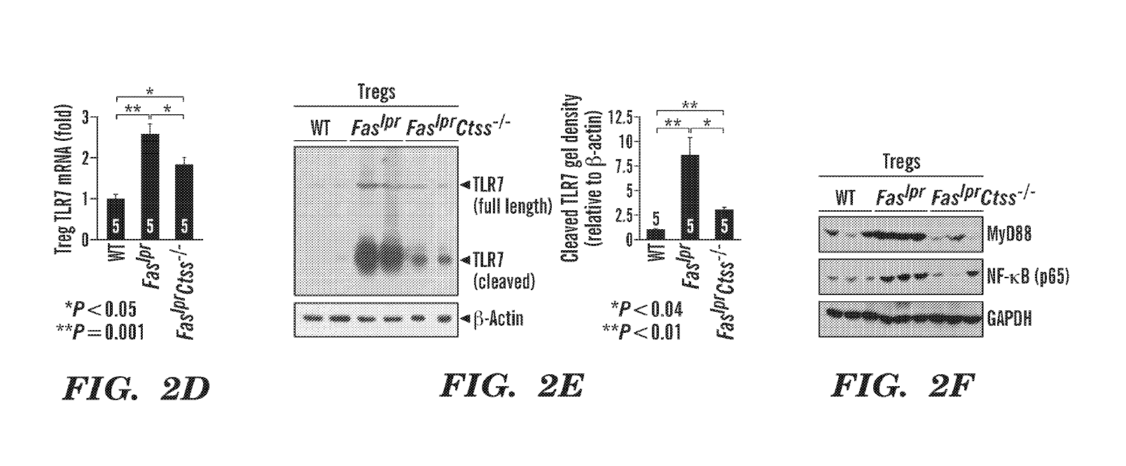

Similar observations were made when lysates from spleen Treg cells from B6-Fas^{lpr} and B6-Fas^{lpr}Ctss^{-/-} mice were used. CD4⁺CD25^{high}Foxp3⁺ Treg cells were purified from crude splenocytes using magnetic beads. FACS analysis showed that the purity of these cells reached to 90% (FIG. 2A). Secondary purification with a cell sorter yielded over 99% pure Treg cells, as assessed by FACS analysis (FIG. 2B) and Foxp3 immunofluorescent staining (data not shown). Treg cells from B6-Fas^{lpr} mice also showed increased CatS activity, as detected by labeling the lysates with the cysteine protease active site JPM probe (FIG. 2C). TLR7 mRNA levels and the 72-kDa cleaved TLR7 fragments were also increased in Treg cells from B6-Fas^{lpr} mice (FIGS. 2D, 2E). CatS deficiency reduced TLR7 mRNA levels (FIG. 2D) and the production of the 72-kDa cleaved TLR7 fragment (FIG. 2E) in Treg cells from B6-Fas^{lpr}Ctss^{-/-} mice. Cleaved TLR7 activates its signaling adaptor MyD88 and downstream NF-κB signaling pathway (25, 26) that is responsible for the production of inflammatory cytokines such as IL6, TNF-α, and IL17, all which affect Treg differentiation and immunosuppressive activity (27-29). Immunoblot analysis of Treg lysates revealed increased activation of MyD88 and NF-κB. Both p-MyD88 and p-NF-κB (p60) levels were increased in Treg isolated from the spleens of B6-Fas^{lpr} mice. CatS deficiency reduced the activation of both p-MyD88 and p-p60 in Treg cells from B6-Fas^{lpr}Ctss^{-/-} mice (FIG. 2F).

CatS Controls Treg Differentiation and Function.

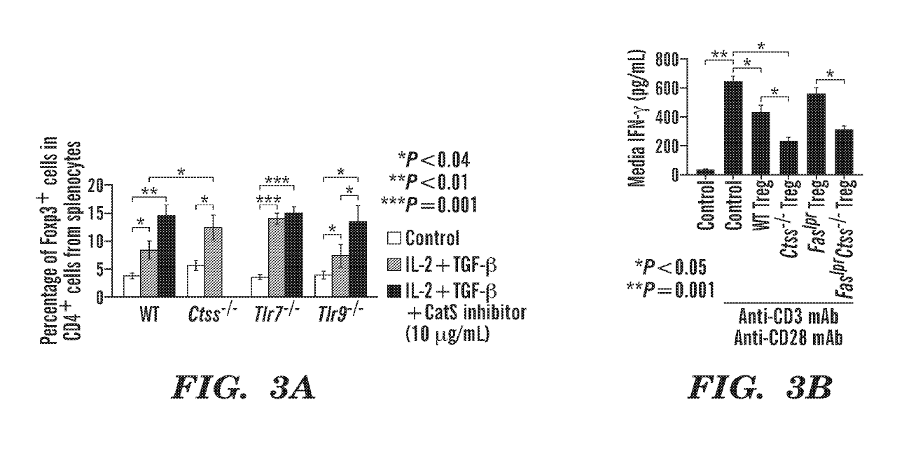

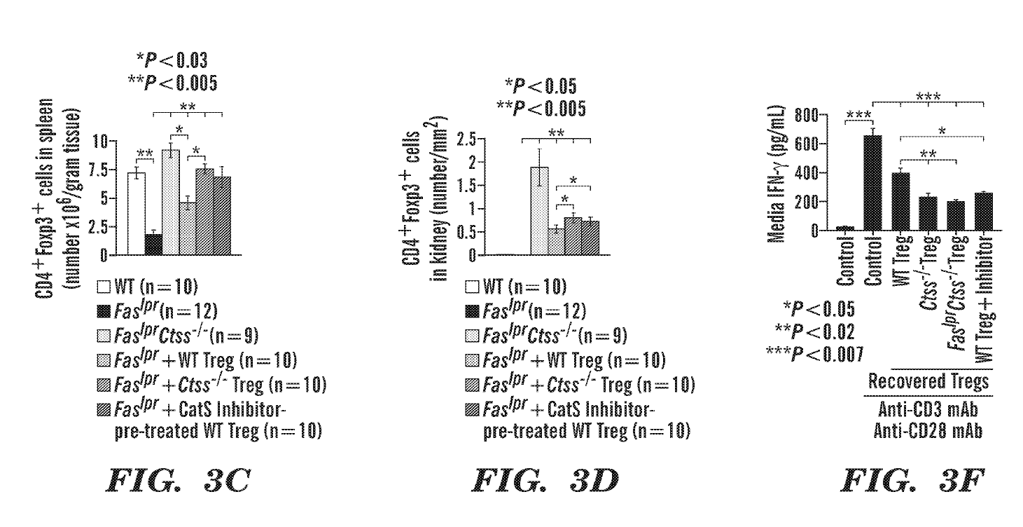

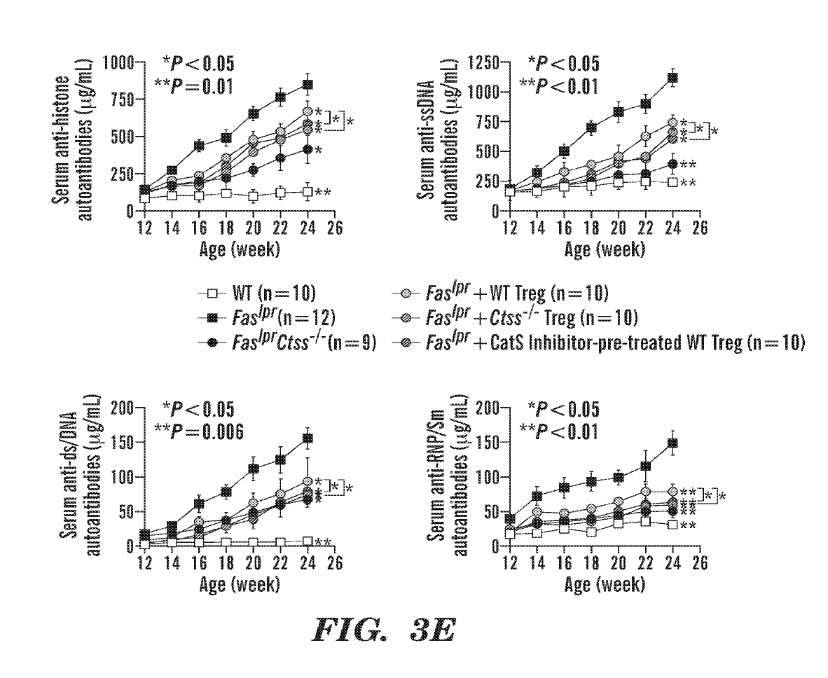

Increased TLR7 expression and activation, elevated MyD88 and NF-κB phosphorylation in kidneys and Treg cells from lupus-prone B6-Fas^{lpr} mice are consistent with increased IL6 (30) and IL17 (31) in SLE patients and lupus-prone mice. Elevated serum IL6 and IL17 and reduced serum IL2 and TGF-β were found in B6-Fas^{lpr} mice but reduced serum IL6 and IL17 and increased serum IL2 and TGF-β were found in B6-Fas^{lpr}Ctss^{-/-} mice further indicating a role of CatS in Treg biology (data not shown). CatS deficiency and inhibition resulted in enhanced Treg differentiation as determined by the expression of Foxp3 when total spleen cells were cultured in the presence of IL2 and TGF-β (FIG. 3A). Splenocytes from WT or Tlr9^{-/-} mice showed identical Treg differentiation patterns in the presence of IL2 and TGF-β. IL2/TGF-β-induced Treg differentiation increased further by 80%˜100% in cells obtained from Ctss^{-/-} mice or from B6 normal or Tlr9^{-/-} mice treated with the CatS inhibitor. In contrast, IL2/TGF-β-induced Treg differentiation of splenocytes from Tlr7^{-/-} mice was comparable to CatS inhibitor-treated cells from WT or Tlr9^{-/-} mice. CatS inhibition did not change the Treg levels in Tlr7^{-/-} splenocytes (FIG. 3A), suggesting a role of CatS in TLR7 action and Treg differentiation, and that TLR9 plays no role in IL2/TGF-β-induced Treg differentiation.

CD4⁺CD25^{high}Foxp3⁺ Treg cells prepared from splenocytes from B6-Ctss^{-/-} or B6-Fas^{lpr}Ctss^{-/-} mice using magnetic beads were 50%˜60% more potent than their WT counterparts in suppressing CD4⁺CD25⁻ Teff cell function as estimated by IFN-γ production (FIG. 3B). Circulating Treg cell numbers are reduced in SLE patients and lupus-prone mice (32-34). Using FACS analysis, significantly fewer Treg cells were detected in the spleens of B6-Fas^{lpr} mice compared to WT control mice. CatS deficiency increased spleen Treg to levels comparable to those in control mice (FIG. 3C).

Reduced spleen Treg numbers in B6-Fas^{lpr} mice increased 15 weeks after adoptive transfer of in vitro prepared spleen Treg cells from WT mice. When the same numbers of donor Treg cells from Ctss^{-/-} mice or WT Treg cells pre-incubated overnight with a CatS-selective inhibitor were transferred into B6-Fas^{lpr} mice, elevated spleen Treg cells were found 15 weeks later (FIG. 3C). Using CD4 and Foxp3 immunofluorescent double staining, negligible Treg cells were detected in the kidneys from WT or B6-Fas^{lpr} mice. CatS-deficiency increased the numbers of Treg cells in the kidney tissue. Adoptive transfer of WT Treg cells also increased the numbers of kidney Treg cells and significantly more kidney Treg cells were detected when donor Treg cells were derived from Ctss^{-/-} mice or from WT mice and pre-incubated overnight with a CatS-selective inhibitor (FIG. 3D, representative data are shown to the right).

Starting at 12 weeks of age, B6-Fas^{lpr} mice developed autoantibodies against histone, ssDNA, dsDNA, and RNP/Sm and probably other autoantigens (FIG. 3E) and autoantibody titers were reduced significantly in the B6-Fas^{lpr}Ctss^{-/-} mice (data not shown). It was asked whether Treg cells procured in the absence of CatS could suppress autoantibody production. Adoptive transfer of Treg cells from WT mice reduced serum autoantibody titers, but Treg cells obtained from Ctss^{-/-} mice or from WT mice and pre-incubated overnight with a CatS inhibitor prior to transfer resulted in significantly stronger suppression of autoantibody titers against all four tested autoantigens (FIG. 3E). Spleen Treg cells isolated from WT mice significantly suppressed Teff cell activity as determined by the production of IFN-γ production. Treg cells obtained from B6-Fas^{lpr}Ctss^{-/-} mice, or from B6-Fas^{lpr} mice 15 weeks after they had received Treg cells from Ctss^{-/-} mice or from B6-Fas^{lpr} mice 15 weeks after they had received cells from WT mice and pre-incubated overnight with the CatS inhibitor displayed significantly higher immunosuppressive activity against Teff cells (FIG. 3F).

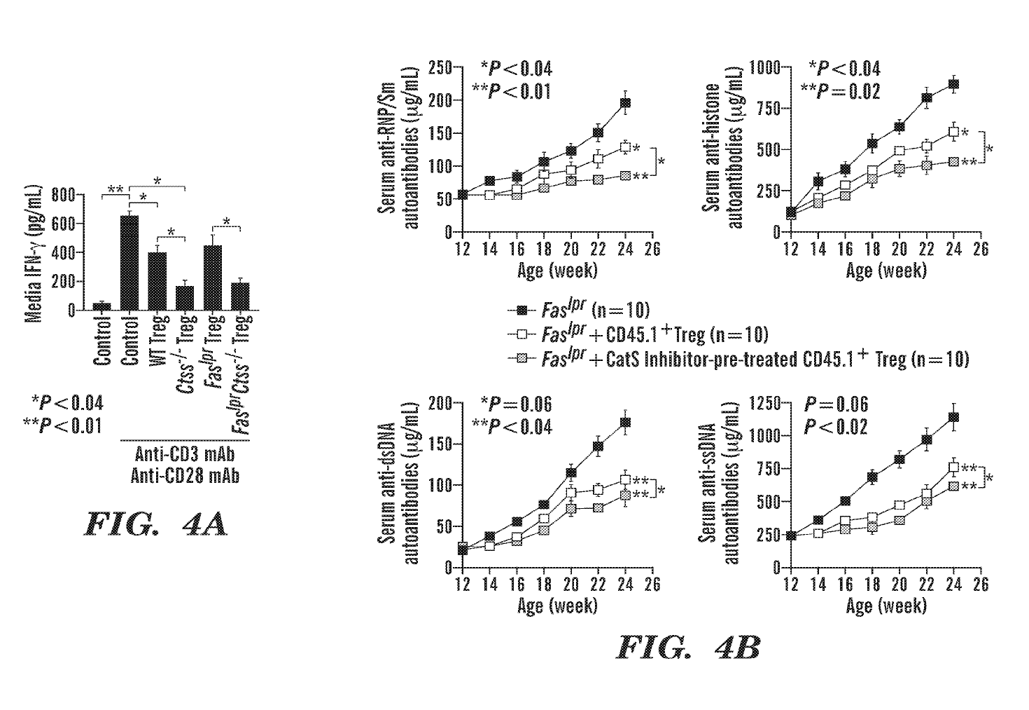

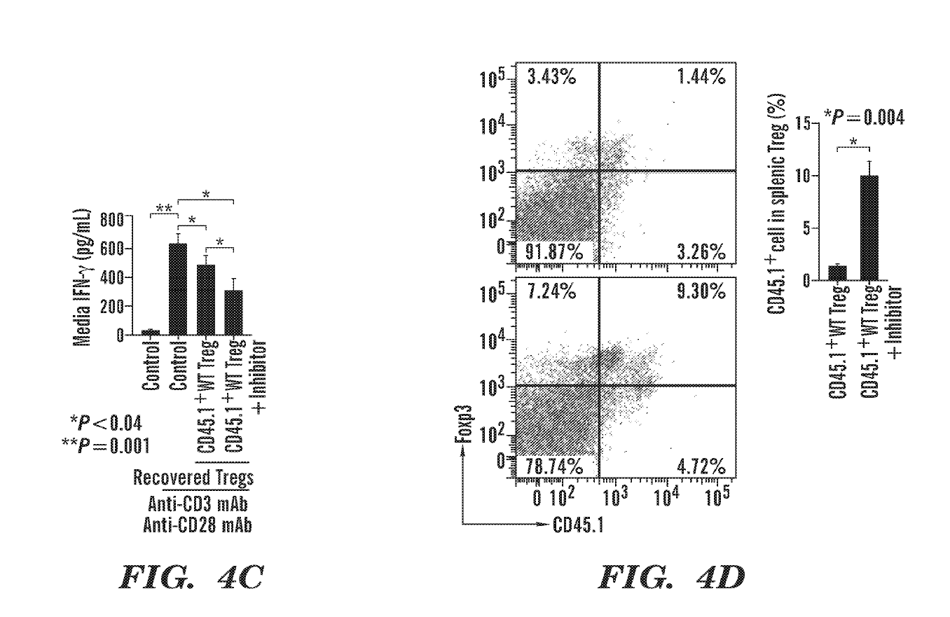

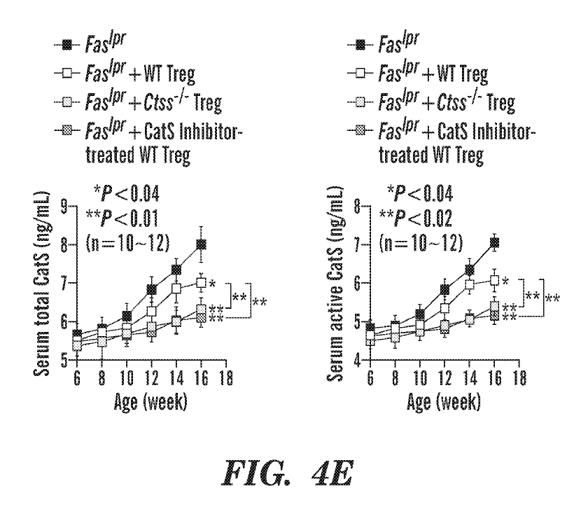

Spleen Treg cells purified in two steps—magnetic bead separation followed by secondary cell sorting purification (FIG. 2B), display the same immunosuppressive activity (FIG. 4A) to those purified in one-step—magnetic bead separation (FIG. 3B) when tested for their ability to suppress anti-CD3/CD28 mAb-mediated Teff cell IFN-γ production. Treg cells from Ctss^{-/-} or B6-Fas^{lpr}Ctss^{-/-} mice showed consistently higher immunosuppressive activity than those from WT or B6-Fas^{lpr} mice.

To trace the survival and immunosuppressive activity of Treg cells in B6-Fas^{lpr} mice, CD45.1⁺ Treg cells were purified using the two-step approach (magnetic beads and cell sorter) to ensure Treg purity (FIG. 2B). CD45.1⁺ Treg cells also reduced serum autoantibody titers when transferred into B6-Fas^{lpr} mice. Overnight pre-incubation of Treg cells with a CatS inhibitor prior to transfer significantly increased donor CD45.1⁺ Treg activity as manifested by the reduction of serum autoantibody titers in the recipient B6-Fas^{lpr} mice (FIG. 4B). After 15 weeks, CatS inhibitor-pre-treated CD45.1⁺ Treg cells retained higher immunosuppressive activity against Teff cells as compared that of untreated CD45.1⁺ Treg cells (FIG. 4C). Of note, both FACS analysis (CD45.1⁺Foxp3⁺) and CD45.1 immunostaining detected about 10 times more CD45.1⁺ donor Treg cells in the spleens on mice which had received Treg cells pretreated with the CatS inhibitor compared to those which had received non-treated Treg cells (FIG. 4D). CD45.1 and Ki67 immunofluorescent co-staining demonstrated that a brief treatment of donor Treg cells in vitro with a CatS inhibitor led to sustained Treg survival and proliferation in vivo (data not shown). Reduced lupus manifestations in B6-Fas^{lpr} mice receiving Treg cells was associated with a reduction of serum total and active CatS concentrations. CatS-deficient and CatS inhibitor-treated donor Treg cells caused a significant reduction of serum CatS in recipient mice compared to mice which had received non-treated Treg cells (FIG. 4F).

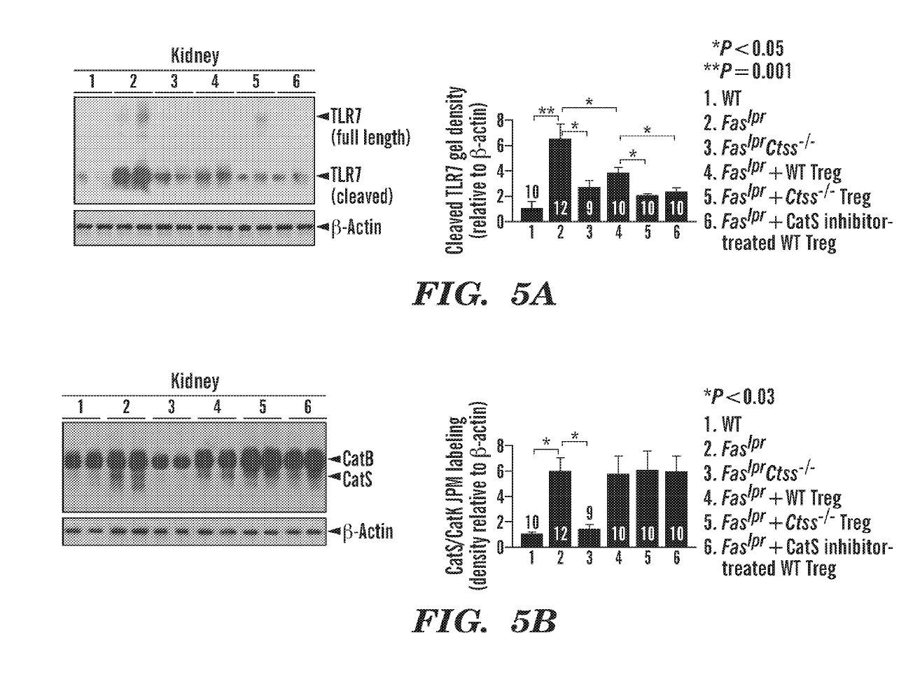

TLR7 immunoblot analysis demonstrated that cleaved TLR7 was significantly reduced in kidney lysates from B6-Fas^{lpr} mice receiving WT Treg cells and further reduced in kidneys from those receiving Treg cells from CatS-deficient or CatS inhibitor pre-treated Treg cells, reaching the levels recorded for lysates from B6-Fas^{lpr}Ctss^{-/-} mice (FIG. 5A). Interestingly, JPM probe labeling detected comparable levels of CatS activity in kidney lysates from B6-Fas^{lpr}-recipient mice regardless the Treg type they had received (FIG. 5B), suggesting that donor Treg cells did not affect tissue CatS activity.

CatS Controls Human Treg Differentiation, Survival, and Immunosuppressive Activity.

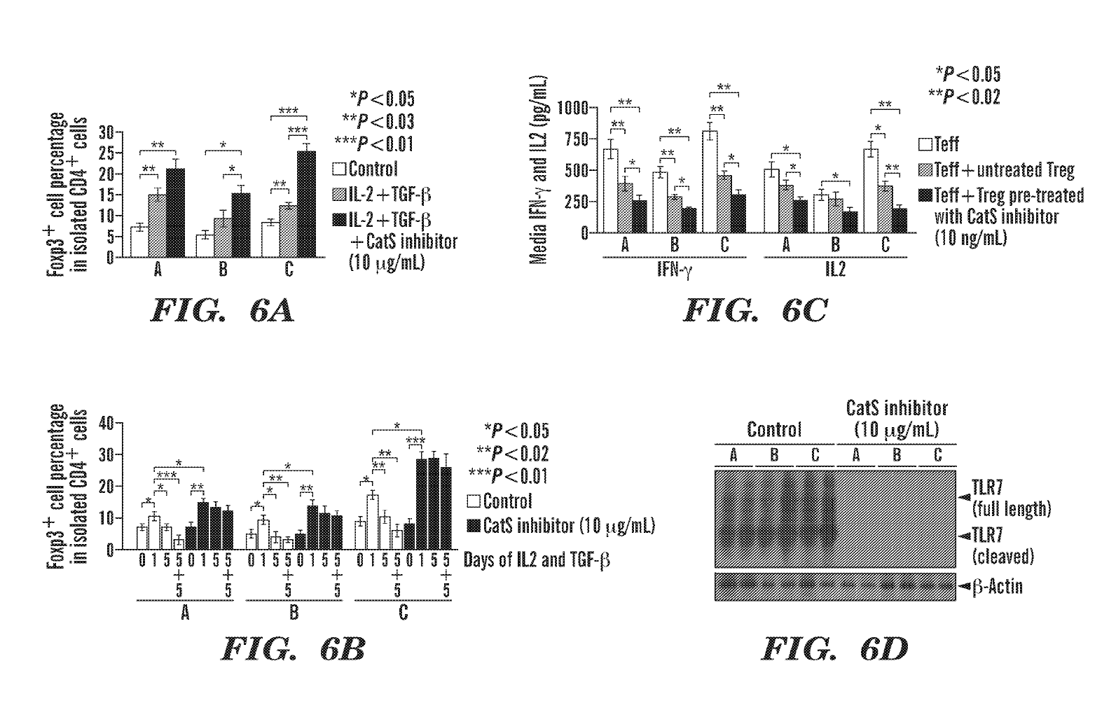

CatS inhibitor-treated Treg cells can provide effective cell therapy for human SLE and other autoimmune diseases associated with Treg insufficiency. Naïve CD4⁺ T cells were purified from PBMCs obtained from healthy individuals and cultured in vitro in the presence of IL2, TGF-β, and a CatS inhibitor. As shown in FIG. 6A, CatS inhibition resulted in increased IL2/TGF-β-induced Treg differentiation among cells from all three donors by 40%˜100%. IL2/TGF-β-induced human Treg differentiation was transient and survived less than 5 days. CatS inhibition enhanced IL2/TGF-β-induced Treg differentiation by additional 40˜80%, as determined by FACS analysis, and prolonged the Treg lifespan for 5 days and an additional 5 days without IL2 and TGF-β (FIG. 6B). CatS inhibition also increased the immunosuppressive activity of human Treg cells when co-cultured with CD4⁺CD25⁻ Teff cells. Overnight pre-treatment of doubly purified CD4⁺CD25^{high}Fopx3⁺ Treg cells from all three donors with the CatS inhibitor prior to co-culture with Teff cells resulted in significantly decreased production of both IL2 and IFN-γ by an additional 30%˜50% (FIG. 6C). Finally, as it was the case with mouse Treg cells (FIGS. 2E-2F), culture of human PBMCs with a CatS inhibitor decreased profoundly TLR7 expression, as determined by TLR7 immunoblot analysis (FIG. 6D). Longer exposure of the immunoblot revealed much less cleaved TLR7 fragments from CatS inhibitor-treated PBMCs than those from untreated PBMCs (data not shown).

Discussion

Described herein is the demonstration that CatS is involved in the regulation of the immune system though yet another distinct mechanism, which involves the deterioration of Treg function by activating TLR7. Previously, it was shown that CatS mediates CD74 processing and MHC-II peptide loading in APC endolysosomes to control CD4⁺ T-cell activation and antibody production (1-3). While MHC class-II-antigenic peptide complex formation and antigen presentation in APCs from mice with the H-2^{b} (e.g. C57BL/6 mice) and H-2^{d} (e.g. Balb/C mice) haplotypes depend on CatS activity in CD74 processing (3, 35), the same action does not take place in APCs from mice with the H-2^{k} (e.g. C3H/He and MRL/MpJ mice), H-2^{s} (e.g. SJL/J mice), and H-2U (e.g. PL/J mice) haplotypes (9, 10). After noticing that CatS deficiency (accompanying manuscript) or pharmacologic inhibition (ref. 36 and accompanying manuscript) suppresses disease in lupus-prone mice irrespectively of their H-2 haplotypes, described herein is another mechanism whereby this protease interferes with immunoregulation: it deteriorates Treg function by activating TLR7.

Like antigen processing, CatS-mediated TLR7 activation, initially reported in macrophages (11, 12), occurs also in the endolysosomes. It is demonstrated herein that this activity takes place in mouse and human Treg cells as well as murine kidneys. The identification of non-H-2-restricted mechanism of action of CatS has significant implications in the treatment of autoimmune diseases in patients who have an obviously polymorphic MHC class-II system (37).

One unexpected discovery of this study is that in human and mouse naïve CD4⁺ T cells and splenocytes, CatS deficiency or inhibition enhanced IL2/TGF-β-induced Treg differentiation by 40% to 100% and prolonged Treg lifespan. A brief (overnight) inhibition in vitro with a CatS inhibitor prolonged Treg survival in lupus-prone mice for 15 weeks or possibly longer, without loosing their greater than 50% more potent immunosuppressive activity against Teff cells. CD4⁺CD25^{high}Foxp3⁺ Treg cells mediate peripheral tolerance and suppress excessive immune responses (32). Besides CD4⁺CD25⁻ Teff cells, Treg cells also suppress macrophages, B cells, NK cells, and CD8⁺ T cells (38, 39). Patients with active SLE have fewer Treg cells in lymphoid organs, the kidneys, or the blood. In addition to number changes, Treg cells in the peripheral tissues from SLE patients also show reduced immunosuppressive activity (34, 40). The lupus-prone mice (BZB×ZNW)F1 and MRL/MpJ-Fas^{lpr} are also known to have similar reductions in Treg numbers and immunosuppressive activity (41, 42). Treg numbers increase in patients with inactive disease (40) or after treatment (43, 44), indicating that increasing their numbers and function can be therapeutic.

It is demonstrated herein that long-lived effective Treg cells can be generated in vitro and infused into patients to control disease. It is specifically contemplated herein that Treg cells harvested from patients with autoimmune diseases can be treated in vitro with a CatS inhibitor and reinfused to control disease. Such an approach would obviate the administration of CatS inhibitors to patients and save them from potential side effects.

### REFERENCES

1. Riese R J, Wolf P R, Bromine D, Natkin L R, Villadangos J A, Ploegh H L, et al. Essential role for cathepsin S in MHC class II-associated invariant chain processing and peptide loading. Immunity. 1996; 4(4):357-66.
2. Hsing L C, Rudensky A Y. The lysosomal cysteine proteases in MHC class II antigen presentation. Immunol Rev. 2005; 207:229-41.
3. Shi G P, Villadangos J A, Dranoff G, Small C, Gu L, Haley K J, et al. Cathepsin S required for normal MHC class II peptide loading and germinal center development. Immunity. 1999; 10(2):197-206.
4. Saegusa K, Ishimaru N, Yanagi K, Arakaki R, Ogawa K, Saito I, et al. Cathepsin S inhibitor prevents autoantigen presentation and autoimmunity. J Clin Invest. 2002; 110(3):361-9.
5. Baugh M, Black D, Westwood P, Kinghorn E, McGregor K, Bruin J, et al. Therapeutic dosing of an orally active, selective cathepsin S inhibitor suppresses disease in models of autoimmunity. J Autoimmun. 2011; 36(3-4):201-9.
6. Hsing L C, Kirk E A, McMillen T S, Hsiao S H, Caldwell M, Houston B, et al. Roles for cathepsins S, L, and B in insulitis and diabetes in the NOD mouse. J Autoimmun. 2010; 34(2):96-104.
7. Yang H, Kala M, Scott B G, Goluszko E, Chapman H A, Christadoss P. Cathepsin S is required for murine autoimmune myasthenia gravis pathogenesis. J Immunol. 2005; 174(3): 1729-37.
8. van Bavel C C, Fenton K A, Rekvig O P, van der Vlag J, Berden J H. Glomerular targets of nephritogenic autoantibodies in systemic lupus erythematosus. Arthritis Rheum. 2008; 58(7):1892-9.
9. Riese R J, Shi G P, Villadangos J, Stetson D, Driessen C, Lennon-Dumenil A M, et al. Regulation of CD1 function and NK1.1(+) T cell selection and maturation by cathepsin S. Immunity. 2001; 15(6):909-19.
10. Villadangos J A, Riese R J, Peters C, Chapman H A, Ploegh H L. Degradation of mouse invariant chain: roles of cathepsins S and D and the influence of major histocompatibility complex polymorphism. J Exp Med. 1997; 186(4):549-60.
11. Ewald S E, Lee B L, Lau L, Wickliffe K E, Shi G P, Chapman H A, et al. The ectodomain of Toll-like receptor 9 is cleaved to generate a functional receptor. Nature. 2008; 456(7222):658-62.
12. Park B, Brinkmann M M, Spooner E, Lee C C, Kim Y M, Ploegh H L. Proteolytic cleavage in an endolysosomal compartment is required for activation of Toll-like receptor 9. Nat Immunol. 2008; 9(12):1407-14.
13. Uematsu S, Akira S. Toll-like receptors and Type I interferons. J Biol Chem. 2007; 282(21):15319-23.
14. Christensen S R, Shupe J, Nickerson K, Kashgarian M, Flavell R A, Shlomchik M J. Toll-like receptor 7 and TLR9 dictate autoantibody specificity and have opposing inflammatory and regulatory roles in a murine model of lupus. Immunity. 2006; 25(3):417-28.
15. Wu X, Peng S L. Toll-like receptor 9 signaling protects against murine lupus. Arthritis Rheum. 2006; 54(1):336-42.
16. Zhang R, Xing M, Ji X, Gu L, Yang X, Wang H, et al. Interferon-alpha and interleukin-6 in SLE serum induce the differentiation and maturation of dendritic cells derived from CD34+ hematopoietic precursor cells. Cytokine. 2010; 50(2): 195-203.
17. Fujimoto M, Nakano M, Terabe F, Kawahata H, Ohkawara T, Han Y, et al. The influence of excessive IL-6 production in vivo on the development and function of Foxp3+ regulatory T cells. J Immunol. 2011; 186(1):32-40.
18. Kelley J, Johnson M R, Alarcon G S, Kimberly R P, Edberg J C. Variation in the relative copy number of the TLR7 gene in patients with systemic lupus erythematosus and healthy control subjects. Arthritis Rheum. 2007; 56(10):3375-8.
19. Blossom S, Chu E B, Weigle W O, Gilbert K M. CD40 ligand expressed on B cells in the BXSB mouse model of systemic lupus erythematosus. J Immunol. 1997; 159(9):4580-6.
20. Pisitkun P, Deane J A, Difilippantonio M J, Tarasenko T, Satterthwaite A B, Bolland S. Autoreactive B cell responses to RNA-related antigens due to TLR7 gene duplication. Science. 2006; 312(5780): 1669-72.
21. Kulkarni O P, Sayyed S G, Kantner C, Ryu M, Schnurr M, Sardy M, et al. 4SC-101, a novel small molecule dihydroorotate dehydrogenase inhibitor, suppresses systemic lupus erythematosus in MRL-(Fas)lpr mice. Am J Pathol. 2010; 176(6):2840-7.
22. Shi G P, Munger J S, Meara J P, Rich D H, Chapman H A. Molecular cloning and expression of human alveolar macrophage cathepsin S, an elastinolytic cysteine protease. J Biol Chem. 1992; 267(11):7258-62.
23. Pandiyan P, Zheng L, Ishihara S, Reed J, Lenardo M J. CD4+CD25+Foxp3+ regulatory T cells induce cytokine deprivation-mediated apoptosis of effector CD4+ T cells. Nat Immunol. 2007; 8(12):1353-62.
24. Pawar R D, Patole P S, Zecher D, Segerer S, Kretzler M, Schlondorff D, et al. Toll-like receptor-7 modulates immune complex glomerulonephritis. J Am Soc Nephrol. 2006; 17(1): 141-9.
25. Kawai T, Akira S. The role of pattern-recognition receptors in innate immunity: update on Toll-like receptors. Nat Immunol. 2010; 11(5):373-84.
26. Lau C M, Broughton C, Tabor A S, Akira S, Flavell R A, Mamula M J, et al. RNA-associated autoantigens activate B cells by combined B cell antigen receptor/Toll-like receptor 7 engagement. J Exp Med. 2005; 202(9):1171-7.
27. Pasare C, Medzhitov R. Toll pathway-dependent blockade of CD4+CD25+ T cell-mediated suppression by dendritic cells. Science. 2003; 299(5609):1033-6.
28. Xu L, Kitani A, Fuss I, Strober W. Cutting edge: regulatory T cells induce CD4+CD25−Foxp3− T cells or are self-induced to become Th17 cells in the absence of exogenous TGF-beta. J Immunol. 2007; 178(11):6725-9.
29. Valencia X, Stephens G, Goldbach-Mansky R, Wilson M, Shevach E M, Lipsky P E. TNF downmodulates the function of human CD4+CD25hi T-regulatory cells. Blood. 2006; 108(1):253-61.
30. Studnicka-Benke A, Steiner G, Petera P, Smolen J S. Tumour necrosis factor alpha and its soluble receptors parallel clinical disease and autoimmune activity in systemic lupus erythematosus. Br J Rheumatol. 1996; 35(11):1067-74.
31. Wong C K, Lit L C, Tam L S, Li E K, Wong P T, Lam C W. Hyperproduction of IL-23 and IL-17 in patients with systemic lupus erythematosus: implications for Th17-mediated inflammation in auto-immunity. Clin Immunol. 2008; 127(3):385-93.
32. Sakaguchi S. Naturally arising Foxp3-expressing CD25+CD4+ regulatory T cells in immunological tolerance to self and non-self. Nat Immunol. 2005; 6(4):345-52.
33. Suen J L, Li H T, Jong Y J, Chiang B L, Yen J H. Altered homeostasis of CD4(+) FoxP3(+) regulatory T-cell subpopulations in systemic lupus erythematosus. Immunology. 2009; 127(2):196-205.
34. Bonelli M, Savitskaya A, von Dalwigk K, Steiner C W, Aletaha D, Smolen J S, et al. Quantitative and qualitative deficiencies of regulatory T cells in patients with systemic lupus erythematosus (SLE). Int Immunol. 2008; 20(7):861-8.
35. Kala M, Chen C R, McLachlan S M, Rapoport B, Aliesky H, Chapman H A. Cathepsin S is not crucial to TSHR processing and presentation in a murine model of Graves' disease. Immunology. 2005; 116(4):532-40.
36. Rupanagudi K V, Kulkarni O P, Lichtnekert J, Darisipudi M N, Mulay S R, Schott B, et al. Cathepsin S inhibition suppresses systemic lupus erythematosus and lupus nephritis because cathepsin S is essential for MHC class II-mediated CD4 T cell and B cell priming. Ann Rheum Dis. 2015; 74(2):452-63.
37. Alper C A, Larsen C E, Dubey D P, Awdeh Z L, Fici D A, Yunis E J. The haplotype structure of the human major histocompatibility complex. Hum Immunol. 2006; 67(1-2):73-84.
38. Miyara M, Sakaguchi S. Natural regulatory T cells: mechanisms of suppression. Trends Mol Med. 2007; 13(3):108-16.
39. Langier S, Sade K, Kivity S. Regulatory T cells: the suppressor arm of the immune system. Autoimmun Rev. 2010; 10(2):112-5.
40. Miyara M, Amoura Z, Parizot C, Badoual C, Dorgham K, Trad S, et al. Global natural regulatory T cell depletion in active systemic lupus erythematosus. J Immunol. 2005; 175(12):8392-400.
41. Scalapino K J, Tang Q, Bluestone J A, Bonyhadi M L, Daikh D I. Suppression of disease in New Zealand Black/New Zealand White lupus-prone mice by adoptive transfer of ex vivo expanded regulatory T cells. J Immunol. 2006; 177(3):1451-9.
42. Parietti V, Monneaux F, Decossas M, Muller S. Function of CD4+,CD25+ Treg cells in MRL/lpr mice is compromised by intrinsic defects in antigen-presenting cells and effector T cells. Arthritis Rheum. 2008; 58(6):1751-61.
43. Suarez A, Lopez P, Gomez J, Gutierrez C. Enrichment of CD4+ CD25high T cell population in patients with systemic lupus erythematosus treated with glucocorticoids. Ann Rheum Dis. 2006; 65(11):1512-7.
44. Sfikakis P P, Souliotis V L, Fragiadaki K G, Moutsopoulos H M, Boletis J N, Theofilopoulos A N. Increased expression of the FoxP3 functional marker of regulatory T cells following B cell depletion with rituximab in patients with lupus nephritis. Clin Immunol. 2007; 123(1):66-73.

### Example 2: Cathepsin S Inhibition Changes Regulatory T-Cell Activity in Regulating Bladder Cancer and Immune Cell Proliferation and Apoptosis

Regulatory T cells (Tregs) are immune suppressing cells, but their roles in tumor growth have been elusive, depending on tumor type or site. As described in Example 1, demonstrated herein is a role of cathepsin S (CatS) in reducing Treg immunosuppressive activity. The effect of CatS inhibition in Tregs on tumors was investigated. Mice receiving inhibitor-treated Tregs had fewer splenic and tumor Tregs, and lower levels of tumor and splenic cell proliferation than those in mice received saline-treated Tregs. In vitro, inhibitor-treated Tregs showed lower proliferation and higher apoptosis than saline-treated Tregs when cells were exposed to mouse bladder carcinoma MB49 cells. In contrast, both types of Tregs showed no difference in proliferation when they were co-cultured with normal splenocytes. Inhibitor-treated Tregs had less apoptosis in splenocytes, but more apoptosis in splenocytes with MB49 conditioned media than saline-treated Tregs. In turn, less proliferation and more apoptosis of MB94 cells was detected after co-culture with inhibitor-treated Tregs, compared with saline-treated Tregs. B220⁺ B-cell, CD4⁺ T-cell, and CD8⁺ T-cell proliferation and apoptosis were also lower in splenocytes co-cultured with inhibitor-treated Tregs than with saline-treated Tregs. Under the same condition, addition of cancer cell conditioned media greatly increased CD8⁺ T-cell proliferation and reduced CD8⁺ T-cell apoptosis. These observations indicate that CatS inhibition of Tregs reduces overall T-cell immunity under normal conditions, but enhances CD8⁺ T-cell immunity in the presence of cancer cells.

CD4⁺CD25⁺Foxp3⁺ regulatory T cells (Tregs) are immunosuppressive cells that play protective role in inflammatory diseases, such as atherosclerosis, abdominal aortic aneurysms, obesity and diabetes (1-7). In contrast, Tregs suppress cytotoxic CD8⁺ T cells in many solid tumors, thereby having a significant negative effect on tumor-associated overall survival (8). In patients with ovarian cancers, tumor Tregs exhibited more potent suppression of CD8⁺ T cells than those in the peripheral (9). Reduced tumor Treg contents were associated with improved overall survival of these patients (10). Tregs in gastric tumors (11), in the peripheral blood from patients with B cell lymphoma (12) or acute B lymphoblastic leukemia (13) were all elevated. In patients with breast cancer, tumor Treg contents and expression of PD-L1 (programmed death ligand 1) were positively correlated (14). In a mouse model of lung adenocarcinoma, Tregs in the advanced lung tumors suppressed the anti-tumor T-cell responses. Depletion of Tregs caused immune-mediated tumor destruction (15). In mouse melanoma cell tumor model, Treg-specific depletion of PTEN (phosphatase and tensin homolog), which stabilizes Tregs, reduced tumor growth and inflammation (16). In B-cell acute lymphoblastic leukemia or 4T1 mammary carcinoma mice, Treg ablation led to CD8⁺ T-cell generation, tumor regression, and extended survival (17, 18). All these studies point to a detrimental role of Tregs in cancers. However, Tregs also exert no role or even opposite role in different types of tumors. For example, in patients with multiple myeloma, the effect of anti-tumor drug bortezomib treatment was associated with Treg expansion. Treg ex vivo expansion decreased multiple myeloma viability (19). In colorectal cancers, Treg infiltration indicated better prognosis (20-23). In head and neck or oesophageal cancers, the progonostic role of Tregs was highly influenced by tumor site, and correlated with the molecular subtype and tumor stage (8). Therefore, the role of Tregs in tumors can be complicated and may depend on the types of the tumors.

Bladder carcinoma is the fifth most common cancer with increased incidence worldwide (24, 25). Patients with high content of tumor infiltration of Tregs may have elevated incidence of recurrence (26), although a direct role of Tregs in bladder cancer has not been tested. Of note, the role of Tregs in inflammatory diseases or in cancers may vary depending on the subtypes of Tregs. A recent study reported a detrimental role of fat-resident Tregs that contribute to age-associated insulin resistance. Selective depletion of this Treg population increased adipose tissue insulin sensitivity (27). In colorectal cancers, tumor infiltration of non-immunosuppressive Foxp3^{lo} Tregs with no expression of the naive T cell marker CD45RA and instability of Foxp3 showed better prognosis than immunosuppression-competent Foxp3^{hi} Tregs (16). Therefore, either depletion of Foxp3^{hi} Tregs or local increase of Foxp3^{lo} Tregs suppressed or prevented tumor formation.

It is described herein that Tregs had increased immunosuppressive activity after a brief treatment with a small molecule inhibitor of cathepsin S (CatS), a lysosomal cyeteine protease that mediates lysosomal protein proteolysis. CatS participates in toll-like receptor-7 (TLR7) activation in Tregs, thereby changing the TLR7 downstream signaling and cytokine profile leading to elevated immunosuppressive activity. It was investigated whether Tregs with or without CatS inhibition affect bladder tumor cells in a mouse bladder cancer MB49 cell subcutaneous implantation model.

Materials and Methods

Mice, Tumor Cell Culture, and Tumor Model

Wild-type (WT) C57BL/6 mice and CD45.1 transgenic mice (C57BL/6) were purchased from the Jackson Laboratory (Bar Harbor, Me.). MB49 cells are chemically induced murine bladder carcinoma cell line derived from C57BL/6 male mice (28) (American Type Culture Collection, ATCC, Manassas, Va.). MB49 cells were maintained in RPMI 1640 medium (Gibco, Big Cabin, Okla.) supplemented with 10% fetal calf serum, 10 mM HEPES (4-(2-hydroxyethyl)-1-piperazineethanesulfonic acid), penicillin (100 IU/ml) and streptomycin (100 μg/ml), 5×10⁻⁵ M 2-mecaptoethnal, and 2 mM L-glutamine. Each WT recipient mouse received subcutaneous implantation of 2×10⁶ MB49 cells on the right flank.

Treg Purification and Adoptive Transfer.

To perform Treg adoptive transfer in mice with tumor, splenic CD4⁺CD25⁺ Treg cells were purified from C57BL/6 WT or C57BL/6 CD45.1 transgenic mice according to the manufacturer's instructions (Miltenyi Biotec, Inc., Auburn, Calif.). The resulting CD45.1⁺CD4⁺CD25⁺ Treg cells were also further purified with cell sorter (The BD FACSAira™ Cell Sorter, BD Biosciences, San Jose, Calif.). Treg purity was confirmed by FACS and anti-Foxp3 antibody-mediated immunofluorescent staining. WT Treg cells were incubated with a CatS inhibitor (10 μg/mL, see, e.g., data available on the world wide web at clinicaltrials.gov/show/NCT01515358) overnight before adoptive transfer. Each 9-week-old male C57BL/6 WT mouse received intravenous injection of 5×10⁶ donor Treg cells three days after mice received MB49 cell subcutaneous implantation. On day 7, mice were sacrificed, splenocytes and tumor tissue single cell preparation were analyzed for CD4, CD25 and Foxp3 by FACS. Spleen and tumor tissue were also collected to prepare 5 μm frozen sections for immunohistochemical analysis.

Immunohistochemistry.

Frozen tumor and spleen sections were prepared for immunohistochemical staining using FITC-conjugated anti-mouse CD45.1 monoclonal antibody (1:1000, Abcam, Cambridge, Mass.), anti-mouse Ki67 monoclonal antibody (1:400, Thermo Fisher Scientific, Waltham, Mass.), CD31 (1:1500, BD Biosciences). Frozen tumor and spleen sections were also prepared for histological detection of apoptotic cells using the terminal deoxynucleotidyl transferase-mediated dUTP nick-end labeling (TUNEL) assay kit according to the manufacturer (EMD Millipore, Billerica, Mass., USA).

Treg Cell Co-Cultures.

CD4⁺CD25⁺ Tregs were isolated from total CD4⁺ T cells in spleens from CD45.1⁺ transgenic mice using a CD4⁺CD25⁺ Treg isolation kit according to the manufacturer's instructions (Miltenyi Biotec, Cambridge, Mass.). FACS analysis confirmed the purity of each preparation of greater than 93%. CD4⁺CD25⁺ Tregs (1×10⁶) were treated with a CatS inhibitor (10 ug/ml) or phosphate-buffered saline (PBS) for 24 hours. Mouse bladder carcinoma MB49 cells were cultured in RPMI 1640 complete medium to 90% confluence. Splenocytes were isolate from C57BL/6 WT mice. To assess the interactions between Tregs and MB49 tumor cells or with TW splenocytes, tumor cells and WT splenocytes were collected and added to Tregs on a 6-well plate at a 1:1 ratio. For splenocyte and Treg co-cultures, MB49 tumor cell conditioned media was also added. After 24 hours of co-culture, Tregs or MB49 tumor cells were collected, washed, and used for FACS analysis.

Flow Cytometry of Tregs in Tumors and Spleens from MB49-Implanted Mice.

Mouse splenocytes were prepared by removing red blood cells as described previously (29). To determine the proportion of CD4⁺CD25⁺Foxp3⁺ Treg cells in splenocytes and tumor cell preparation, 100 μl of splenocyte or tumor total cell suspension (˜1×10⁷ cell) was incubated at 4° C. in PBS containing 2% FCS with the Alexa Fluor 488-conjugated anti-CD4 and PE-conjugated anti-CD25 fluorescent monoclonal antibodies (mAb), followed by Alexa Fluor 647-conjugated Foxp3 intracellular staining. Staining for intracellular Foxp3 was performed using the fixation/permeabilization solution kit (BD Biosciences). Isotype controls were used for each antibody. The following antibodies were used for FACS analysis: FcR-blocking antibody anti-CD16/32 mAb (eBioscience, San Diego, Calif.), Alexa Fluor 488-conjugated anti-CD4 mAb, PE-conjugated anti-CD25 mAb, Alexa Fluor 647-conjugated anti-mFoxp3 mAb, anti-mouse-PE and all isotype controls (all from BD Biosciences).

Flow Cytometry of Co-Cultured Cells.

For the analysis of splenocytes that were co-cultured with different CD45.1⁺ Tregs, total cell mixtures, containing splenocytes, Tregs, with and without MB49 conditioned media were incubated with APC-conjugated anti-mouse CD45.1 mAb and PE-conjugated anti-CD4 mAb, PE-conjugated anti-CD8 mAb, or PE-conjugated anti-B220 mAb at 4° C. for 30 min for cell surface staining then suspended at a density of 2×10⁶ cells/mL in PBS containing 1% FBS. For Treg analysis, cells were incubated with APC-conjugated anti-mouse CD45.1 mAb.

Cell proliferation was detected by Ki67 staining. To a cell pellet (1-5×10⁷ cells), 5 ml of cold 70%-80% ethanol was added drop wise, followed by incubation at −20° C. for 2 hours. A 30-40 ml of a wash buffer (PBS with 1% FBS and 0.09% NaN₃, pH7.2) was added to the fixed cells. Cells were precipitated by centrifugation for 10 minutes at 1000 rpm, washed once with 30˜40 ml of a wash buffer. Cells were resuspended to a concentration of 1×10⁷/ml (1×10⁶/100 μl) and transferred 100 μl of cell suspension into a fresh tube, followed by adding 20 μl of properly diluted antibody and 10 μl of propidium iodide staining solution (BD Bioscience). After a gentle mix, tubes were incubated at room temperature for 20-30 minutes in dark, washed with 2 ml of a washing buffer at 1000 rpm for 5 minutes, and suspended into 0.5 ml of PBS for FACS analysis.

Cell apoptosis was assessed using a FITC Annexin V Apoptosis Detection Kit I (RUO) according to the manufacturer (BD Bioscience). Cells were washed twice with a cold PBS and then suspended in lx binding buffer, at a concentration of 1×10⁶ cells/ml. Each 100 μl of the suspension containing 1×10⁵ cells was transferred to a 5-ml culture tube, followed by adding 5 μl of FITC-Annexin V and 10 μl of propidium iodide staining solution. After a gentle vortex, cells were incubated for 15 min at room temperature in dark, followed by adding 400 μl of 1× binding buffer for FACS analysis within 1 hour. Flow cytometric acquisition was performed using a FACSCalibur™ (BD Immunocytometry Systems), and all analyses were performed using Flowjo™ software (Tree Star Inc, Ashland, Oreg.).

Statistics.

Because of relatively small sample sizes and sometime skewed data distribution, the non-parametric Mann-Whitney U test was selected for paired data sets and one-way ANOVA with post-hoc Bonferroni test was used for comparison among three or more groups to examine statistical significance for all data from cultured cells and mouse model. P<0.05 was considered statistically significant. All analyses were performed using R software, version 3.0.1.

Results

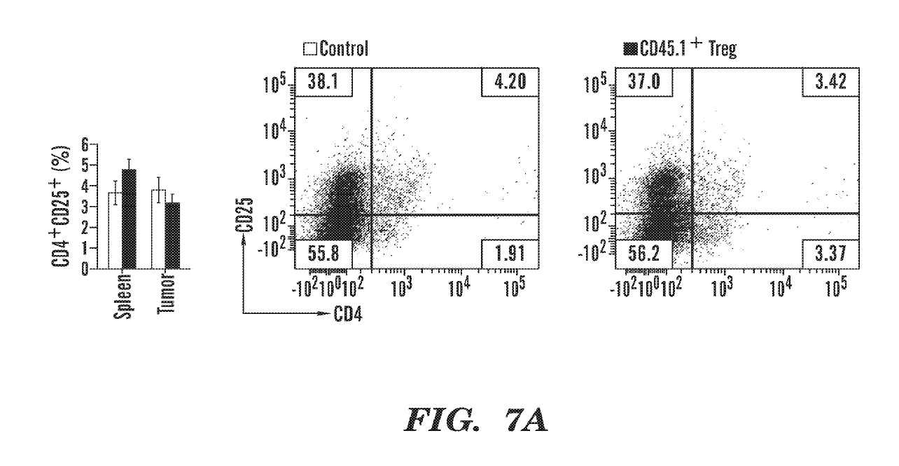

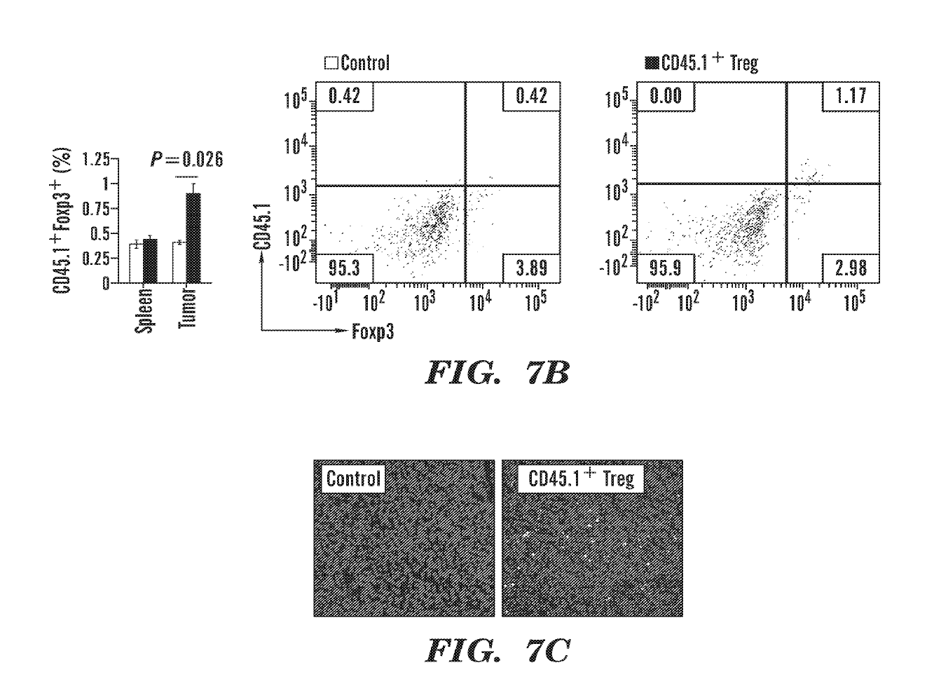

Treg adoptive transfer has been used to assess Treg immunobiology in multiple mouse disease models (5, 7). To mice that received subcutaneous implantation of MB49 bladder cancer cells, intravenous Tregs from CD45.1 transgenic mice were also given to trace donor cells. Four days after Treg adoptive transfer, splenic and tumor total CD4⁺CD25⁺ Tregs and donor CD45.1⁺Foxp3⁺ cells were assessed by FACS analysis. Treg adoptive transfer did not change significantly splenic and tumor total CD4⁺CD25⁺ Tregs (FIG. 7A). Negligible donor CD45.1⁺Foxp3⁺ Treg cells were detected in spleens from recipient mice. However, donor CD45.1⁺Foxp3⁺ Treg cells were detected in tumors (FIG. 7B). FITC anti-mouse CD45.1 mouse monoclonal antibody-mediated immunofluorescent staining also detected CD45.1-positive cells in tumors from recipient mice that were given CD45.1⁺Foxp3⁺ Treg cells (FIG. 7C).

CatS Inhibitor-Treated Tregs Reduced Recipient Mouse Splenic and Tumor Cell Tregs and Total Cell Proliferation.

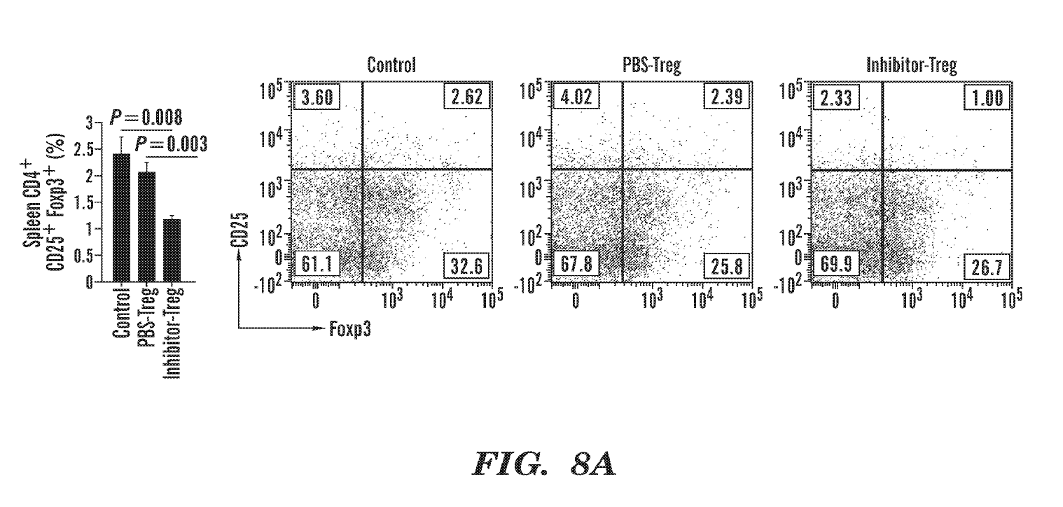

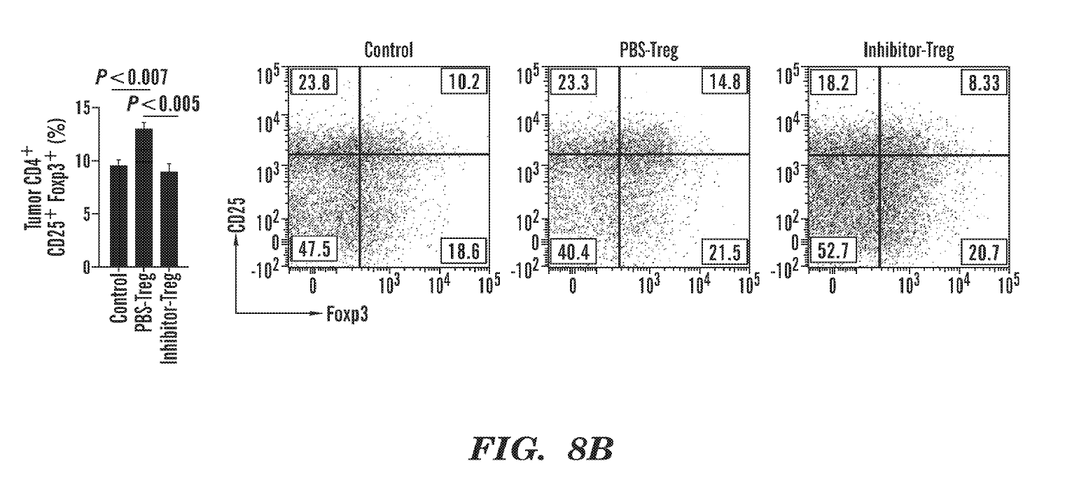

FACS analysis of splenocytes from recipient mice with subcutaneous implantation of MB49 tumor cells did not reveal significant differences in CD4⁺CD25⁺Foxp3⁺ total Treg contents between mice received with or without donor PBS-treated Tregs. However, splenic CD4⁺CD25⁺Foxp3⁺ Tregs were significantly reduced in mice received inhibitor-treated Tregs (FIG. 8A). Consistent with increased donor Tregs in tumors (FIG. 7B), significant increase of CD4⁺CD25⁺Foxp3⁺ Tregs in tumors from mice received adoptive transfer of PBS-treated donor Tregs was also detected. However, tumor CD4⁺CD25⁺Foxp3⁺ Treg cells were significantly reduced in recipient mice that received inhibitor-treated Tregs, to the level of control mice without Treg adoptive transfer (FIG. 8B).

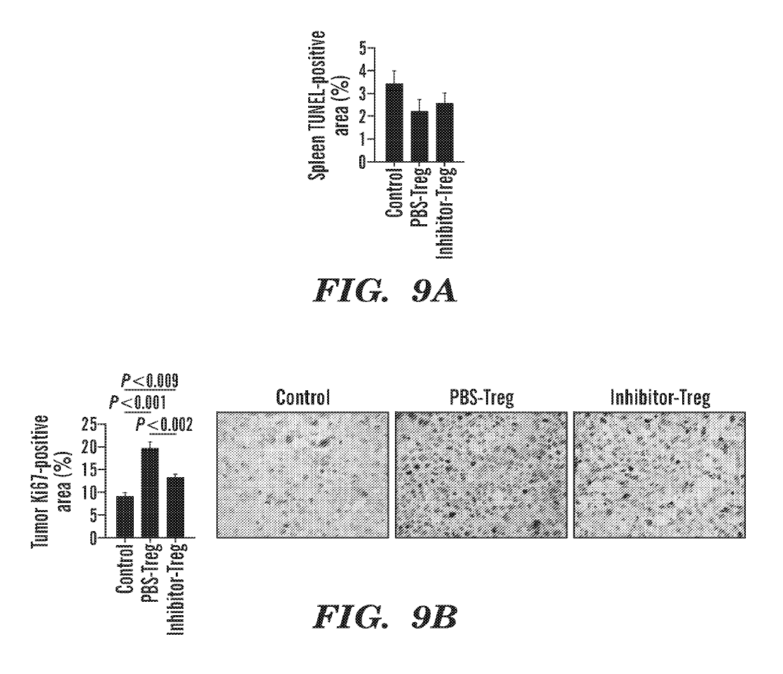

To test why inhibitor-treated donor Treg cells reduced splenic and tumor Tregs, immunohistochemical analysis of both spleens and tumors from control mice without Treg adoptive transfer and from mice received with PBS- or inhibitor-treated Tregs were performed. Total TUNEL-positive areas in percentage in spleens did not differ among all three groups of mice (FIG. 9A). However, significant larger areas of Ki67-positive cells were detected in tumors (FIG. 9B) and spleens (FIG. 9C) in recipient mice that received PBS-treated Tregs than those from control mice. In contrast, when inhibitor-treated Tregs were used, Ki67-positive cell areas were significantly reduced, compared with those from mice receiving PBS-treated Tregs (FIGS. 9B, 9C). These observations indicate a role of CatS inhibitor-treated Tregs in regulating tumor and splenic cell proliferation. In both tumors and spleens, adoptive transfer of PBS-treated Tregs did not change the CD31-positive microvessel numbers. However, adoptive transfer of inhibitor-treated Tregs increased the microvessel numbers in the tumor tissues, but not in the spleens (FIG. 9D).

Differential Roles of CatS Inhibitor-Treated Tregs in the Presence and Absence of Tumor Cells.

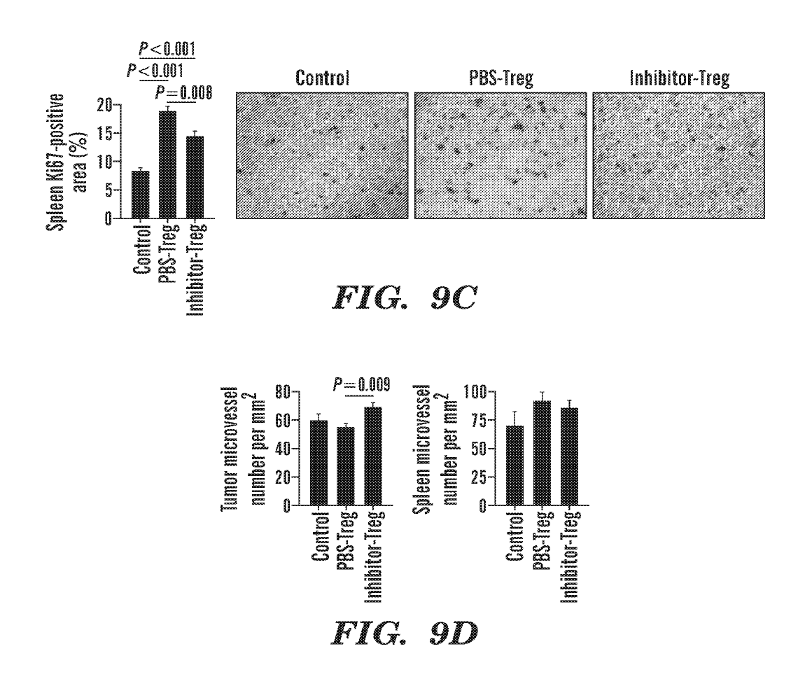

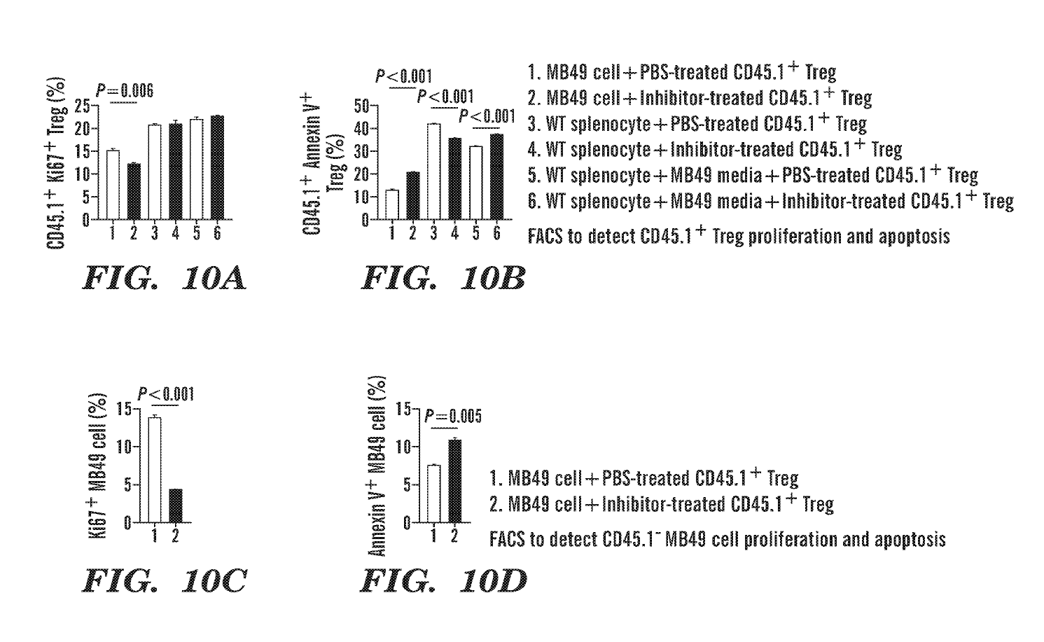

CatS inhibitor-treated Tregs showed much stronger immunosuppressive activity than untreated or PBS-treated Tregs to T effector cells. Inhibitor-treated Tregs reduced splenic and tumor CD4⁺CD25⁺Foxp3⁺ Tregs in recipient mice (FIGS. 8A-8B), and these cells exerted stronger activity than PBS-treated Tregs in inhibiting splenic and tumor cell proliferations in mice bearing the MB49 bladder tumors (FIGS. 9B, 9C). All these observations pointed to a hypothesis that CatS inhibitor-treated Tregs differ from PBS-treated Tregs in controlling cell growth and possibly cell death in tumor-bearing mice. Inhibitor-treated Tregs may present different immunobiological activities under tumoral conditions from those under normal conditions. To test these hypotheses, PBS- and CatS inhibitor-treated Tregs were cultured with MB49 cells, or total splenocytes from WT mice, or splenocytes together with MB49 cell conditioned media for 24 hours. When Tregs were co-cultured with MB49 tumor cells, inhibitor-treated Tregs showed reduced proliferation (CD45.1⁺Ki67⁺) but increased apoptosis (CD45.1⁺Annexin V⁺). In contrast, when Tregs were co-cultured with WT splenocytes, the difference of Treg proliferation disappeared between the groups, and inhibitor-treated Treg cell apoptosis reduced (FIGS. 10A-10B). In the presence of WT splenocytes and MB49 tumor cell conditioned media, Treg proliferation remained no significant difference between the groups, but inhibitor-treated Treg cell apoptosis became higher than PBS-treated Tregs, a pattern similar to that from splenocytes co-cultured with tumor cells (FIGS. 10A, 10B). These observations indicate that CatS inhibitor-treated Tregs exert different activities (proliferation and apoptosis) under different conditions, in tumor tissues (MB49 or its conditioned media) or in normal tissues (WT splenocytes).

In turn, different Tregs also affected differently tumor cell proliferation and apoptosis. Co-culture of inhibitor-treated Tregs showed significantly more suppression of tumor cell proliferation (Ki67⁺ MB49 cells) (FIG. 10C) and much higher tumor cell apoptosis (Annexin V⁺ MB49 cells) (FIG. 10D) than that with PBS-treated Tregs.

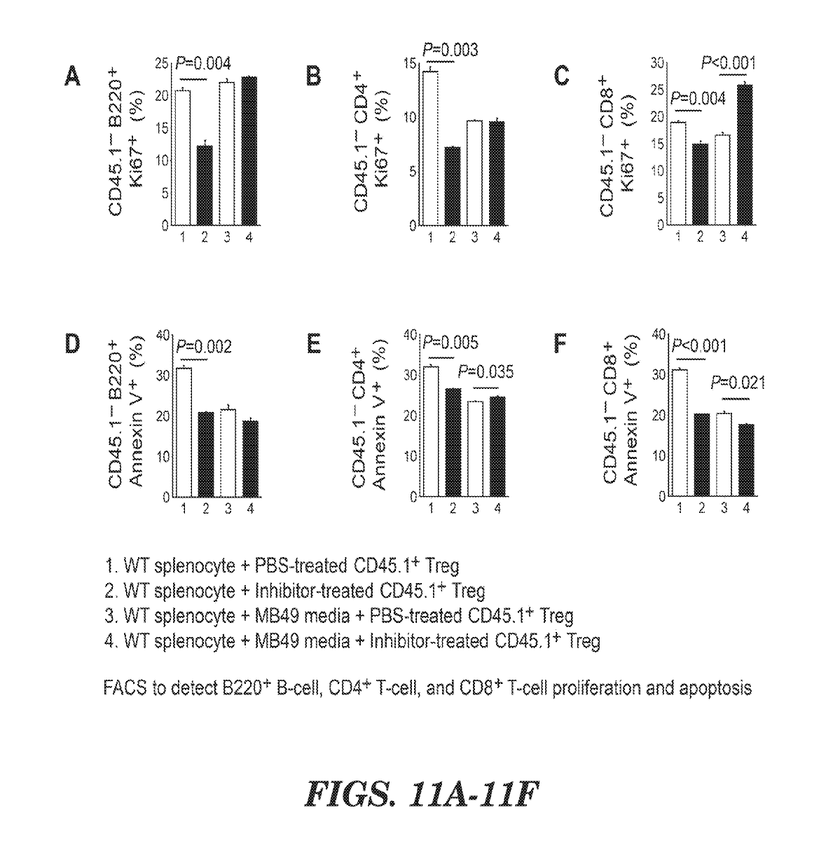

When inhibitor-treated Tregs and PBS-treated Tregs were co-cultured with WT splenocytes, the activities of these Tregs in regulating B-cell, CD4⁺ T-cell, and CD8⁺ T-cell proliferation and apoptosis also differed, depending on the Treg type and the presence of tumor cell conditioned media. In the absence of tumor cells, inhibitor-treated Tregs showed less activity on the proliferation and apoptosis of B220⁺ B cells, CD4⁺ T cells, and CD8⁺ T cells in WT splenocytes (FIGS. 11A, 11B) than PBS-treated Tregs. In contrast, when splenocyte and Treg co-cultures were cultured in the presence of MB49 tumor cell conditioned media, inhibitor-treated Tregs showed no difference from PBS-treated Tregs in B220⁺ B-cell (B220⁺Ki67⁺) and CD4⁺ T-cell (CD4⁺Ki67⁺) proliferation (FIGS. 11A, 11B). However, co-culture of inhibitor-treated Tregs showed much higher CD8⁺ T-cell (CD8⁺Ki67⁺) proliferation than that of PBS-treated Tregs when co-cultures were carried in the presence of MB49 tumor cell conditioned media. B220⁺ B-cell apoptosis (B220⁺Annexin V⁺) did not differ when co-cultures were performed in the presence of MB49 conditioned media. CD4⁺ T cells showed more apoptosis (CD4⁺Annexin V⁺) and CD8⁺ T cells showed less apoptosis (CD8⁺Annexin V⁺) when splenocytes were co-cultured with inhibitor-treated Tregs than those were co-cultured with PBS-treated Tregs in the presence of MB49 tumor cell conditioned media (FIGS. 11E, 11F). These observations indicate that CatS inhibition of Tregs increased Treg immunosuppression activity (reduced B-cell, CD4⁺ T-cell, and CD8⁺ T-cell proliferation) under normal conditions (WT splenocytes), as expected. However, CatS inhibition can enhance Treg immunocompetent activity by increasing CD8⁺ T-cell proliferation when Tregs are exposed to a tumoral environment.

Discussion

No role for Tregs in promoting tumor growth was detected in mouse bladder carcinoma cell subcutaneous implant tumor model, affirming a tumor type-dependent role of Tregs in affecting tumor growth. Demonstrated herein are unexpected observations that may change the view of Tregs and their immunobiological activities.

Adoptive transfer of CatS inhibitor-treated Tregs reduced recipient spleen and tumor Treg contents. These observations may be explained by overall reduced spleen and tumor cell proliferation. In vitro, a role of CatS inhibition of Tregs in reducing tumor cell proliferation was confirmed. Tumor cells, in turn, reduced Treg proliferation and increased Treg apoptosis after Tregs were pre-treated with a CatS inhibitor.

In vitro studies revealed a role of tumor cells in enhancing Treg apoptosis after Tregs were pre-treated with a CatS inhibitor. In turn, inhibitor-treated Tregs were more potent than PBS-treated Tregs in promoting tumor cell apoptosis. These in vitro data indicate more cell apoptosis in spleens and tumors from mice received inhibitor-treated Tregs than those from mice received PBS-treated Tregs.

Interaction between Tregs and other immune cells has been a major focus of Treg studies. Under normal conditions, Tregs play an important role in suppressing T effector cells. CatS inhibition enhances this activity of Tregs. Indeed, significantly lower levels of B220⁺ B-cell, CD4⁺ T-cell, and CD8⁺ T-cell proliferation were demonstrated when WT splenocytes were co-cultured with CatS inhibitor-treated Tregs than in those were co-cultured with PBS-treated Tregs. These observations agree with the observation that CatS inhibition can increase Treg immunosuppressive activity, possibly by reducing the numbers of B cells and T cells via reduced proliferation and/or increased apoptosis of these lymphocytes. However, in the presence of tumor cell conditioned media, such activity of inhibitor-treated Tregs became reversed. Although the proliferations of B cells and CD4⁺ T cells were comparable whether WT splenocytes were co-cultured with PBS- or inhibitor-treated Tregs in the presence of tumor cell conditioned media. Under this condition, CatS inhibitor-treated Tregs greatly enhanced CD8⁺ T-cell proliferation and reduced CD8+ T-cell apoptosis. Without wishing to be bound by theory, it is possible that CatS inhibition may increase CD8⁺ T-cell immunity in mice with tumors because of increased proliferation and decreased apoptosis of CD8⁺ T cells.

Together, this study demonstrates that CatS inhibition can turn Tregs into potent immunosuppressive cells in the absence of tumor cells. However, in the presence of tumor cells or even tumor cell conditioned media, CatS inhibition can turn Tregs into immunoactive or immunocompetent cells by enhancing the number of cytotoxic CD8⁺ T cells and reducing the apoptosis of this T-cell population. It is contemplated herein that CatS inhibitor-treated Tregs can be utilized in a new regimen for therapy of tumors.

### REFERENCES

1. Ait-Oufella H1, Tedgui A2. Regulatory T-Cell Plasticity: Another Layer of Complexity in Atherosclerosis. Circ Res. 2016 May 13; 118(10):1461-3.
2. Yildiz A, Gur M. CD4+ CD25+ regulatory T-cells in acute coronary syndromes. Eur Heart J. 2007 March; 28(6):774-5.
3. Meng X1, Yang J1, Dong M1, Zhang K1, Tu E2, Gao Q3, Chen W2, Zhang C1, Zhang Y1. Regulatory T cells in cardiovascular diseases. Nat Rev Cardiol. 2016 March; 13(3): 167-79.
4. Yin M1, Zhang J, Wang Y, Wang S, Böckler D, Duan Z, Xin S. Deficient CD4+CD25+ T regulatory cell function in patients with abdominal aortic aneurysms. Arterioscler Thromb Vasc Biol. 2010 September; 30(9):1825-31.
5. Zhou Y1, Wu W2, Lindholt JS3, Sukhova GK4, Libby P4, Yu X5, Shi GP6. Regulatory T cells in human and angiotensin II-induced mouse abdominal aortic aneurysms. Cardiovasc Res. 2015 Jul. 1; 107(1):98-107.
6. Eller K1, Kirsch A, Wolf A M, Sopper S, Tagwerker A, Stanzl U, Wolf D, Patsch W,
Rosenkranz A R, Eller P. Potential role of regulatory T cells in reversing obesity-linked insulin resistance and diabetic nephropathy. Diabetes. 2011 November; 60(11):2954-62.
7. Feuerer M1, Herrero L, Cipolletta D, Naaz A, Wong J, Nayer A, Lee J, Goldfine A B, Benoist C, Shoelson S, Mathis D. Lean, but not obese, fat is enriched for a unique population of regulatory T cells that affect metabolic parameters. Nat Med. 2009 August; 15(8):930-9.
8. Shang B1, Liu Y2, Jiang SJ2, Liu Y2. Prognostic value of tumor-infiltrating FoxP3+ regulatory T cells in cancers: a systematic review and meta-analysis. Sci Rep. 2015 Oct. 14; 5:15179.
9. Bu M1, Shen Y2, Seeger WL3, An S1, Qi R1, Sanderson JA3, Cai Y4. Ovarian carcinoma-infiltrating regulatory T cells were more potent suppressors of CD8(+) T cell inflammation than their peripheral counterparts, a function dependent on TIM3 expression. Tumour Biol. 2016 March; 37(3):3949-56.
10. Knutson KL1,2, Maurer MJ3, Preston CC4, Moysich KB5, Goergen K3, Hawthorne KM3, Cunningham JM6, Odunsi K5, Hartmann LC7, Kalli KR7, Oberg AL3, Goode EL8. Regulatory T cells, inherited variation, and clinical outcome in epithelial ovarian cancer. Cancer Immunol Immunother. 2015 December; 64(12):1495-504.
11. Kindlund B1, Sjöling Å1, Yakkala C1, Adamsson J1, Janzon A1, Hansson LE2, Hermansson M2, Janson P3, Winqvist O3, Lundin SB4. CD4+ regulatory T cells in gastric cancer mucosa are proliferating and express high levels of IL-10 but little TGF-β. Gastric Cancer. 2016 Jan. 18. [Epub ahead of print].
12. Wu W1, Wan J2, Xia R1, Huang Z1, Ni J1, Yang M1. Functional role of regulatory T cells in B cell lymphoma and related mechanisms. Int J Clin Exp Pathol. 2015 Aug. 1; 8(8):9133-9.
13. Ateyah ME1, Hashem ME1, Abdelsalam M2. Epstein-Barr virus and regulatory T cells in Egyptian paediatric patients with acute B lymphoblastic leukaemia. J Clin Pathol. 2016 Jul. 25.
14. Li Z1, Dong P2, Ren M1, Song Y1, Qian X1, Yang Y1, Li S1, Zhang X3, Liu F1. PD-L1 Expression Is Associated with Tumor FOXP3(+) Regulatory T-Cell Infiltration of Breast Cancer and Poor Prognosis of Patient. J Cancer. 2016 Apr. 10; 7(7):784-93.
15. Joshi NS1, Akama-Garren EH1, Lu Y1, Lee DY1, Chang GP1, Li A1, DuPage M1, Tammela T1, Kerper NR1, Farago AF1, Robbins R1, Crowley DM1, Bronson RT2, Jacks T3. Regulatory T Cells in Tumor-Associated Tertiary Lymphoid Structures Suppress Anti-tumor T Cell Responses. Immunity. 2015 Sep. 15; 43(3):579-90.
16. Saito T1,2, Nishikawa H1, Wada H2,3, Nagano Y4, Sugiyama D I, Atarashi K4, Maeda Y1, Hamaguchi M1, Ohkura N1, Sato E5, Nagase H2, Nishimura J2, Yamamoto H2, Takiguchi S2, Tanoue T4, Suda W6, Morita H7, Hattori M6, Honda K4, Mori M2, Doki Y2, Sakaguchi S1. Two FOXP3(+)CD4(+) T cell subpopulations distinctly control the prognosis of colorectal cancers. Nat Med. 2016 June; 22(6):679-84.
17. Manlove LS1, Berquam-Vrieze KE2, Pauken KE3, Williams RT4, Jenkins MK5, Farrar MA6. Adaptive Immunity to Leukemia Is Inhibited by Cross-Reactive Induced Regulatory T Cells. J Immunol. 2015 Oct. 15; 195(8):4028-37.
18. Goudin N1, Chappert P2, Megret J1, Gross DA2, Rocha B2, Azogui O2. Depletion of Regulatory T Cells Induces High Numbers of Dendritic Cells and Unmasks a Subset of Anti-Tumour CD8+CD11c+ PD-1lo Effector T Cells. PLoS One. 2016 Jun. 24; 11(6):e0157822.
19. Ercetin AP1, Ozcan MA2, Aktas S3, Yuksel F4, Solmaz SM4, Sevindik GO4, Katgi A4, Piskin O5, Undar B4. Ex vivo evaluation of the effect of regulatory T cells on the anti-tumor activity of bortezomib in multiple myeloma. Exp Hematol. 2016 April; 44(4):223-30.
20. deLeeuw RJ1, Kost S E, Kakal J A, Nelson B H. The prognostic value of FoxP3+ tumor-infiltrating lymphocytes in cancer: a critical review of the literature. Clin Cancer Res. 2012 Jun. 1; 18(11):3022-9.
21. Sinicrope FA1, Rego R L, Ansell S M, Knutson K L, Foster N R, Sargent D J. Intraepithelial effector (CD3+)/regulatory (FoxP3+) T-cell ratio predicts a clinical outcome of human colon carcinoma. Gastroenterology. 2009 October; 137(4): 1270-9.
22. Salama P1, Phillips M, Grieu F, Morris M, Zeps N, Joseph D, Platell C, Iacopetta B. Tumor-infiltrating FOXP3+ T regulatory cells show strong prognostic significance in colorectal cancer. J Clin Oncol. 2009 Jan. 10; 27(2):186-92.
23. Frey DM1, Droeser R A, Viehl C T, Zlobec I, Lugli A, Zingg U, Oertli D, Kettelhack C, Terracciano L, Tornillo L. High frequency of tumor-infiltrating FOXP3(+) regulatory T cells predicts improved survival in mismatch repair-proficient colorectal cancer patients. Int J Cancer. 2010 Jun. 1; 126(11):2635-43.
24. Watts KL1, Ristau B T, Yamase H T, Taylor J A 3rd. Prognostic implications of lymph node involvement in bladder cancer: are we understaging using current methods? BJU Int. 2011 August; 108(4):484-92.
25. Dighe MK1, Bhargava P, Wright J. Urinary bladder masses: techniques, imaging spectrum, and staging. J Comput Assist Tomogr. 2011 July-August; 35(4):411-24.
26. Parodi A1, Traverso P1,2,3, Kalli F1, Conteduca G1, Tardito S1, Curto M1, Grillo F2,3, Mastracci L2,3, Bernardi C1, Nasi G1, Minaglia F2,3, Simonato A2,3, Carmignani G2,3, Ferrera F1, Fenoglio D1,3,4, Filaci G1,3,4. Residual tumor micro-foci and overwhelming regulatory T lymphocyte infiltration are the causes of bladder cancer recurrence. Oncotarget. 2016 Feb. 9; 7(6):6424-35.
27. Bapat SP1,2, Myoung Suh J2,3, Fang S2,4, Liu S2, Zhang Y1, Cheng A1, Zhou C1, Liang Y1, LeBlanc M2, Liddle C5, Atkins AR2, Yu RT2, Downes M2, Evans RM2,6, Zheng Y1. Depletion of fat-resident Treg cells prevents age-associated insulin resistance. Nature. 2015 Dec. 3; 528(7580):137-41.
28. Summerhayes I C, Franks L M. Effects of donor age on neoplastic transformation of adult mouse bladder epithelium in vitro. J Natl Cancer Inst. 1979 April; 62(4):1017-23.
29. Shi GP1, Villadangos J A, Dranoff G, Small C, Cu L, Haley K J, Riese R, Ploegh H L, Chapman H A. Cathepsin S required for normal MHC class II peptide loading and germinal center development. Immunity. 1999 February; 10(2): 197-206.

### Example 3

Tregs are immunosuppressive cells that control inflammatory diseases, including autoimmune diseases, such as SLE. CatS inhibitor-treated Tregs showed elevated differentiation, immunosuppressive activity, and extended life span. All these activities of Treg are beneficial to SLE, or other autoimmune diseases.

However, since Tregs generally are immunosuppressive cells, some Tregs can promote tumor growth. Therefore, as a therapeutic regimen, some Tregs could be beneficial to autoimmune diseases, but detrimental to cancers. Therefore, it was tested in Example 2 whether elevated immunosuppressive activity of CatS inhibitor-treated Tregs promotes tumor growth. A mouse bladder cancer cell subcutaneous model was used to test this hypothesis. This work demonstrated that CatS inhibitor-treated Tregs have higher immunosuppressive activity than PBS-treated Tregs under normal conditions (co-cultured with wild-type splenocytes). However, under cancer conditions, i.e. co-culture of Tregs with wild-type splenocytes and tumor cells or tumor cell conditioned media, CatS inhibitor-treated Tregs become more immunocompetent than PBS-treated Tregs. CatS inhibitor-treated Tregs showed elevated activity to promote CD8+ T-cell proliferation. These T cells are required to fight cancers.

In conclusion, these findings indicate that, under normal conditions, or non-tumor conditions, CatS inhibitor-treated Tregs are immunosuppressive to autoimmune diseases (as well as other inflammatory diseases such as atherosclerosis, AAA, obesity, and diabetes). However, under tumorous conditions, these cells change their nature into immunocompetent cells against tumor cells by increasing CD8+ T-cell proliferation. Therefore, it contemplated that as a therapeutic regimen, CatS inhibitor-treated Tregs can be beneficial to patients with cancer and/or can be used to treat autoimmune diseases/inflammatory diseases in patients with cancer without exacerbating the cancer.

1. A method of treating a Treg-mediated disease in a subject in need of treatment thereof, the method comprising:
a. contacting a Treg cell ex vivo with an inhibitor of cathepsin S, cathepsin K, and/or cathepsin L; and
b. administering the cell to the subject

2. The method of claim 1, wherein the cell is autologous to the subject.

3. The method of any of claim 1, wherein the Treg-mediated disease is an autoimmune disease; a cancer; a cardiovascular disease; or a metabolic disease.

4. The method of claim 3, wherein the autoimmune disease is selected from the group consisting of:
systemic lupus erthythematosus; type I diabetes; arthritis; Sjoren's syndrome; type-II diabetes; obesity; atherosclerosis; abdominal aortic aneurysm; and transplant rejection. (heart, liver, kidney, skin, lung, etc).

5. The method of claim 1, wherein the inhibitor is an inhibitor of cathepsin S.

6. The method of claim 1, wherein the inhibitor is an inhibitor of cathepsin K.

7. The method of claim 1, wherein the inhibitor is an inhibitor of cathepsin S and cathepsin K.

8. The method of claim 1, wherein the inhibitor is an inhibitor of cathepsin L.

9. The method of claim 1, wherein the inhibitor is an inhibitor of cathepsin S; cathepsin K; and cathepsin L.

10. The method of claim 1, wherein the inhibitor is a small molecule selected from the group consisting of:
LY3000328; odancatib; balicatib; calpeptin; L006235; SID 26681509; VBY-891; VBY-129; VBY-825; and VBY-036.

11. The method of claim 1, wherein the inhibitor is an antibody reagent that binds specifically to cathepsin S, cathepsin K, and/or cathepsin L.

12. The method of claim 1, wherein the cell is contacted with the inhibitor for a period of at least 6 hours.

13. The method of claim 1, wherein the cell is contacted with the inhibitor for a period of no more than 24 hours.

14. (canceled)

15. The method of claim 1, wherein the cells are administered no more frequently than once a month.

16. (canceled)

17. (canceled)

18. The method of claim 1, wherein the subject is not administered an inhibitor of cathepsin S, cathepsin K, and/or cathepsin L.

19. The method of claim 1, wherein the subject is not administered IL-2 or TGF-beta.

20. The method of claim 1, wherein the patient has both a) an autoimmune disease; a cardiovascular disease; or a metabolic disease; and b) a cancer.

21. A composition comprising a Treg cell and at least one inhibitor of cathepsin S, cathepsin K, and/or cathepsin L.

22. The composition of claim 21, wherein the inhibitor is present at a concentration sufficient to increase the activity, proliferation, and/or lifespan of the Treg cell.

23. An engineered Treg cell, the cell having a level of TLR7 polypeptide which is less than 50% of the level found in a naturally-occurring Treg cell.

24.-54. (canceled)
